(19) United States
(12) Patent Application Publication
(10) Pub. No.: US 2023/0134609 A1
(43) Pub. Date: May 4, 2023

(54) SYSTEM AND METHOD FOR TRAINING IN VITRO NEURONS USING HYBRID OPTICAL/ELECTRICAL SYSTEM

(71) Applicant: CCLabs Pty Ltd, Melbourne (AU)

(72) Inventors: Hon Weng Chong, Melbourne (AU); Brett Joseph Kagan, Melbourne (AU); Andy C. Kitchen, Melbourne (AU)

(21) Appl. No.: 18/048,821

(22) Filed: Oct. 21, 2022

Related U.S. Application Data
(60) Provisional application No. 63/273,807, filed on Oct. 29, 2021.

(51) Int. Cl. G06N 3/00; G01N 33/50

(52) U.S. Cl. CPC G06N 3/002; G01N 33/5058

(57) ABSTRACT

A system and method for interfacing a computing device with in vitro biological neurons is described. In one embodiment, a method of interfacing with a plurality of in vitro biological neurons, comprises: generating, by a processing device, a first tensor indicative of a state of a virtual environment; encoding the first tensor into an instruction; generating first signals according to the instruction using a first plurality of electrodes, one or more chemical emitters or one or more light sources; detecting second signals by a second plurality of electrodes, one or more chemical sensors or one or more image sensors, the second signals having been generated by one or more of the plurality of in vitro biological neurons, wherein the second signals represent an action associated with the virtual environment; decoding the second signals into a second tensor; and applying the action to the virtual environment based on the second tensor.

### RELATED APPLICATIONS

This patent application claims the benefit under 35 U.S.C. § 119(e) of U.S. Provisional Application No. 63/273,807, filed Oct. 29, 2021.

### TECHNICAL FIELD

Embodiments of the present disclosure relate, in general, to a neurological computation and experimentation platform.

### BACKGROUND

Silicon computing performance has approximately doubled every 18 months by shrinking transistor size for the last 50 years following Moore's Law. However, since 2015 performance increase has slowed. Transistors are currently at l0nm, and further shrinkage is difficult due to quantum effects. In order to continue technological progress, alternative computation technologies need to be developed to replace silicon-based computing and the Von Nuemann architecture.

At present, a significant amount of funding, more than $26.6 billion USD for start-ups alone, has been devoted to artificial intelligence (AI) research based on using classic computing methods such as machine learning. Current AI approaches such as Deep Learning are often narrow, brittle and require extensive human tuning and design for each task. Even minor variations in a task can break deep neural networks and require retraining. Equally important as performance are the requirements for running these processes. A recent attempt to teach a robot hand to manipulate a Rubik's cube using Reinforcement Learning based AI was successful, but at the cost of 2.8 gigawatts of power. Furthermore, training using Reinforcement Learning approaches are often done in an accelerated simulation to compensate for the relatively long learning time (>100 years equivalent) required. This makes these systems unsustainable to run continuously and unable to respond to dynamically changing scenarios in real-time. In addition to the physical limitations currently facing the producers of silicon chips, it is difficult to see how incremental improvements on transistor density that are reaching a hard limit will solve the problems briefly described here. Despite exuberance about the potential of AI, the actual societal benefits from AI have fallen short of what proponents have hoped.

### BRIEF DESCRIPTION OF THE DRAWINGS

The embodiments described herein will be understood more fully from the detailed description given below and from the accompanying drawings, which, however, should not be taken to limit the application to the specific embodiments, but are for explanation and understanding only.

FIG. 1 illustrates an example system architecture for a biological computing platform, in accordance with one embodiment.

FIG. 2A illustrates an open feedback system, in accordance with one embodiment.

FIG. 2B illustrates a closed-loop feedback system, in accordance with one embodiment.

FIG. 3 illustrates a summary of the free energy principle, whereby an organism will exist in key states, modifying the priors of those states to predict and manipulate the external environment to minimize surprise, in accordance with one embodiment.

FIG. 4 is a sequence diagram illustrating one embodiment for a method of using a biological computing platform.

FIG. 5 is a sequence diagram illustrating one embodiment for a method of providing reinforcement learning to biological neurons in a biological computing platform.

FIG. 6 illustrates an example virtual environment of the game Pong, in accordance with one embodiment.

FIG. 7 illustrates multiple different electrode layout schematics of an MEA having a cell culture thereon, including locations of a sensory area that includes stimulation electrodes, multiple motor regions.

FIG. 8A is a flow diagram illustrating one embodiment for a method of using a biological computing platform.

FIG. 8B is a flow diagram illustrating one embodiment for a method of using a biological computing platform.

FIG. 9 illustrates an example computing device, in accordance with one embodiment.

### DETAILED DESCRIPTION OF THE PRESENT DISCLOSURE

Described herein are embodiments of a biological computing platform usable to perform in vitro training of biological neurons. The biological computing platform may be implemented as a biological computing research platform (also referred to as a neural computation and experimentation platform) that can train in vitro biological neurons into a real- time synthetic biological intelligence (SBI). The biological computing platform may be a biological computing cloud platform that provides network access to biological neural networks (e.g., exposes biological neural network resources through the cloud). In one embodiment, the biological computing platform externalizes networks of biological neurons (e.g., cortical neurons) and provides an interface between the biological neural network and a virtual environment executed on a computing device. Accordingly, the biological computing platform creates an efferent (e.g., vision or other input)/efferent (e.g., motor or other output) loop between the biological neural network and the virtual environment.

Embodiments demonstrate a pure SBI device which adapts behavior to increase performance in a task over time. By embodying these neurons in a virtual environment (e.g., a simulated game world where the outcome of moving a paddle is informed by a direct interpretation of the free energy principle), embodiments show that a neural system will self-organize responsive to training stimuli. In one example, the neural system will self-organize to behave in a way that limits surprising, unpredictable stimulus, and maximizes predictable stimulus. In one example, the neural system will self-organize to behave in a way that ensures continued stimuli (e.g., that avoids situations in which stimuli is withheld). In one example, the neural system will self-organize to behave in a way that maximizes positive feedback and minimizes negative feedback.

Biological neurons are near infinitely scalable, energy efficient (especially as compared to silicon based processors), small, and produce very little heat (e.g., as compared to silicon based processors). For example, a biological neural network in a multi-electrode array (MEA) or other neural processing unit (e.g., a cell excitation and measurement device) has an energy use per synapse of about 2E−10 Joules. In contrast, the energy use per transistor in an example silicon processing device is about 2E−7 Joules. Additionally, biological neural networks are fault tolerant, and in many instances can withstand the destruction of half of the biological neural network and still be able to function. Biological neural networks also exhibit neuroplasticity, which enables highly adaptable intelligence that is suitable for many different applications.

The mechanisms that have been developed to encode and decode artificial neural networks are generally inapplicable to biological neural networks. In an artificial neural network, data is frequently encoded as floating point vectors, which is then input into the artificial neural network. The artificial neural networks then generally are trained to output further vectors, which are easily decodable. However, in a biological neural network, data is encoded as spikes of action potentials (e.g., voltage variation across a cell membrane) of a population of biological neurons. Neural cells communicate using spiking electrical activity via a biological process called an action potential, or more colloquially ‘firing’. During development, cells display distinct patterns of spontaneous activity linked to the physiological maturation at the cell and system level. For cortical cultures from a primary source, activity has been shown to progressively become more stable from approximately two weeks. Cultures differentiated from a pluripotent source may take longer, in some cases with activity beginning around day in-vitro (DIV) 40 and becoming more complex after DIV 80.

In embodiments, the biological computing platform acts as an encoder/decoder to generate signals that can be interpreted by biological neural networks and to decode output signals generated by the biological neural networks.

Making use of the innate computational power of living neurons requires both theoretical and technical advancements, which are discussed in embodiments herein. Embodiments describe a device capable of real-time synthetic biological intelligence (SBI), an integration of biological cortical cells and silicon based traditional computing via a high-density multi-electrode array (MEA). In embodiments, cortical neuronal cells may be differentiated from human induced pluripotent stem cells (hIPSCs) or harvested from embryonic animals such as embryonic mice. These cells form dense connections with rich spiking activity when plated on, for example, a CMOS-based high-density MEA. A closed-loop system may be established to embody these cultures in a virtual environment (e.g., a simulated game-world representing the classic arcade game ‘Pong’) by applying electrical activity as a shared language between neural cells and silicon computing. Leveraging principles derived from the Free Energy Principle (FEP) to direct external stimuli in response to performance of completing one or more tasks (e.g., gameplay performance), statistically significant performance of cortical neurons has been observed. ‘Learning’ was apparent within five minutes of real-time interaction with a virtual environment (e.g., gameplay), seen as a step increase in performance. This indicates the ability for these cultures to self-organize activity in response to relatively sparse information and, therefore, empirical evidence of the innate drive behind biological intelligence. Described herein is a novel biological computing platform, which contrasts with traditional in-silico machine learning approaches by harnessing the unrivalled computational power of neurological systems.

Embodiments provide a biological computing platform that may include a multi-electrode array (MEA) and/or an optics-based equivalent to an MEA and/or a chemical-based equivalent to an MEA that uses optical input and/or output signals connected to a computing device. The MEA, optics-based equivalent to an MEA, chemical-based equivalent of an MEA that uses chemical emitters and/or chemical sensors, and hybrids of MEAs, optics-based equivalents of MEAs and chemical-based equivalents of MEAs are referred to herein as cell excitation and measurement devices, or simply as MEAs. The computing device may be a physical computing device or a virtual computing device. The computing device may execute an interface (referred to herein as an MEA interface though it can also interface with other systems such as a substrate comprising an optics-based or optical system) that enables the computing device to communicate with the MEA and/or other system (and with a biological neural network contained within the MEA and/or other system). The optical system may be referred to as an optical MEA, a phosomilia system, or an optical energy interchange system. The computing device may additionally execute an experiment logic or virtual environment that interfaces with the MEA interface. The MEA interface may receive digital input signals from the experiment logic or virtual environment, convert the digital input signals into instructions for the MEA and/or other system, and then send the instructions to the MEA and/or other system. The instructions may cause the MEA and/or other system to apply a plurality of electrical or optical impulses at excitation sites having coordinates on a 2D grid or other array of excitation sites in the MEA and/or other system. The MEA interface may additionally receive representations of electrical and/or optical signals measured at locations on the 2D grid or other array from the MEA and/or other system, generate responses for the experiment logic or virtual environment based on the representation, and send the responses to the experiment logic or virtual environment. In this manner, the MEA interface enables the virtual environment or experiment logic to interact with the biological neural network on the MEA and/or other system. Some embodiments are discussed with regards to a virtual environment. However, it should be understood that for any such embodiment the virtual environment may be replaced with or supplemented by an experiment logic. Additionally, in embodiments real or physical environments may be used rather than virtual environments. In some embodiments, virtual environments are simulations of real environments.

In one embodiment, a biological computing platform includes an MEA or similar device connected to a computing device. The MEA or similar device may include a two-dimensional (2D) or three-dimensional (3D) grid of excitation sites, a plurality of biological neurons disposed on the MEA or similar device, and a processing device or integrated circuit. Alternatively, the MEA or similar device may be a circuitless chip, which may be connected to a processing device or integrated circuit (e.g., via a printed circuit board). The processing device may be a complementary metal-oxide-semiconductor (CMOS) chip. In one embodiment, the processing device is a component of a system on a chip (SoC) that includes a network adapter, an analog to digital converter and/or a digital to analog converter.

The computing device may receive or generate a digital input signal, convert the digital input signal into instructions for the plurality of electrical, chemical and/or optical impulses, and send the instructions to the MEA and/or other system. The MEA and/or other system may use a digital to analog converter (DAC) to convert the instructions from a digital form into an analog form, and the processing device of the MEA and/or other system may apply the plurality of electrical, chemical and/or optical impulses at excitation sites having coordinates on the 2D grid or other array of excitation sites. In embodiments, optical stimulation designed to elicit an electrical response in cells and electrical stimulation to elicit an electrical response in cells are both referred to as electrical signals. One or more sensors and/or the processing device may measure electrical and/or other signals (e.g., optical signals or chemical signals) output by one or more of the plurality of biological neurons at coordinates of the 2D grid or other array. In embodiments, excitations of neurons may be captured using optical sensors. For example, when neurons fire such firing may be detected optically by one or more optical sensors. Thus, the electrical impulses output by neurons discussed herein may be captured as optical signals that represents an electrical state of the neurons. Accordingly, any discussion herein of electrical signals output by neurons may instead be optical signals detected by one or more optical sensors. The processing device may then generate a representation of the electrical and/or optical signals, and may send the representation back to the computing device. Additionally, any discussion of electrical signals output by neurons may be chemical signals detected by one or more chemical sensors. The computing device may convert the representation into a response readable by a virtual environment or experiment logic, and may send the response to the experiment logic or virtual environment.

In some embodiments, the biological computing platform is a fully optical system that lacks an MEA. Alternatively, the biological computing platform may include an MEA with an optical system that provides optical signals to neurons and/or that receives optical signals from the neurons. It should be understood that embodiments discussed herein with reference to an MEA also apply to alternatives in which a fully optical interface is used rather than an MEA as well as hybrid systems that include an MEA and optical components (e.g., image sensors and/or light sources). The optical interface may perform a similar function as that traditionally performed by an MEA in such embodiments. Accordingly, references to an MEA also apply to optical components that perform a similar function as an MEA. Moreover, any electrical signals discussed herein may be modified such that optical signals are used instead of or in addition to electrical signals, including electrical signals delivered to neurons and electrical signals received from neurons.

In one embodiment, a method of providing a biological computing platform includes receiving a digital input signal from a processing logic. The method further includes converting the digital input signal into instructions for a plurality of electrical, chemical and/or optical impulses, where each electrical, chemical, and/or optical impulse of the plurality of electrical, chemical and/or optical impulses is associated with a two-dimensional (2D) coordinate or three dimensional (3D) coordinate. The method further includes applying the plurality of electrical, chemical and/or optical impulses at specified coordinates of a 2D grid or 3D matrix in a multi-electrode array (MEA) and/or other system (e.g., cell excitation and measurement device) in accordance with the instructions, wherein a plurality of biological neurons are disposed on the MEA and/or other system. The method further includes measuring electrical, chemical and/or optical signals output by one or more of the plurality of biological neurons at one or more additional coordinates of the 2D grid or 3D matrix. The method further includes generating a representation of the one or more electrical, chemical and/or optical signals and sending the representation of the one or more electrical, chemical and/or optical signals to the processing logic.

In one embodiment, a method of interfacing with a plurality of in vitro biological neurons, includes generating, by a processing device, a first tensor indicative of a state of a virtual environment. The first tensor may be encoded into a plurality of electrical potentials, chemical concentrations and/or light intensities, and first electrical signals having the plurality of electrical potentials, chemical signals having the chemical concentrations and/or optical signals having the plurality of light intensities are generated using a first plurality of electrodes, a first plurality of chemical emitters, and/or a first plurality of light sources. Possible coding schemes that may be used include a rate-based coding scheme, a place-based coding scheme, a mixed coding scheme (e.g., that mixes place-based and rate-based coding), and any combination of the above that gives rise to a mixed population based coding scheme wherein the relationship between a plurality of signals for a plurality of neurons is encoded. The method further includes detecting second electrical, chemical and/or optical signals by a second plurality of electrodes, one or more image sensors (e.g., cameras), and/or one or more chemical sensors, the second electrical, chemical and/or optical signals having been generated by one or more of the plurality of in vitro biological neurons. The second electrical, chemical and/or optical signals represent an action associated with the virtual environment. The method further includes decoding the second electrical, chemical and/or optical signals into a second tensor and applying the action to the virtual environment based on the second tensor. Possible coding schemes that may be used for decoding include a rate-based coding scheme, a place-based coding scheme, a mixed coding scheme (e.g., that mixes place-based and rate-based coding), and any combination of the above that gives rise to a mixed population based coding scheme wherein the relationship between a plurality of signals for a plurality of neurons is decoded.

By embedding neurons onto the surface of silicon processing chips that enable the manipulation and measurement of electrophysiological activity, an input/output bridge is established between biological neural networks and computer systems. This gives rise to Synthetic Biological Intelligence (SBI). One challenge in implanting SBI is software integration. To achieve software integration requires significant theoretical advancements in understanding the fundamental basis of neurological intelligence and how it may be manipulated. It is important to understand how the discrete action potential of a single neuron relates to the activity of an assembly of neurons and how that eventually relates to the behavior of an organism in a dynamic environment. The following disclosure describes functioning networks of cortical cells, derived both from primary sources, or differentiated from either human induced pluripotent stem cell (hIPSC) sources, human embryonic pluripotent stem cell (hESC) sources, or any other stem cell source that may give rise to neuronal cell population, onto media such as high-density multi-electrode arrays (HD-MEA) or other substrate suitable for optical interfacing of cells.

Given the compatibility of hardware and cells (wetware), there are still two interrelated processes that can be imagined in a neural system, as required for intelligent behavior. Firstly, the system learns how external states may be influenced by internal states and the outcomes of this influence; secondly, the system infers from the environment when it should adopt a specific state (behavior) in relation to that environment which must be based off a prediction of how that adopted state will influence the environment. To address the first, custom software drivers may be used create low latency closed-loop feedback systems that provide a virtual environment (e.g., simulate a gameplay environment), physical environment (e.g., based on data from real-world sensors), or virtual environment that is a simulation of a physical (i.e., real-world) environment for these biological neural networks (BNNs) through electrical stimulation.

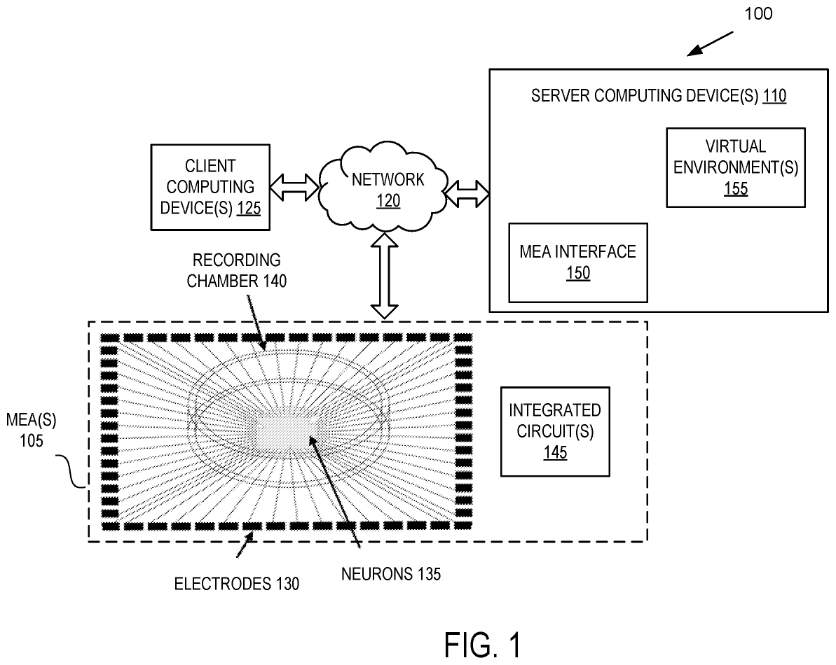

Referring now to the figures, FIG. 1 illustrates an example system architecture for a biological computing platform 100, which may be used to interface with neurons in accordance with one embodiment. As shown, the biological computing platform 100 includes one or more MEA 105 connected to one or more server computing devices 110 via a network 120. The network 120 may be a local area network, a wide area network, a private network (e.g., an intranet), a public network (e.g., the Internet), or a combination thereof. The connection between the MEA(s) 105 and the server computing device(s) 110 may include wired connections, wireless connections, or a combination thereof. Alternatively, the MEA(s) 105 may be directly connected to server computing devices(s) 110 (e.g., via a wired or wireless connection). The MEA(s) 105 may also be a chemical, an optical and/or optical/electrical equivalent of an MEA, which are also referred to as types of MEA(s) herein for convenience.

The server computing devices 110 may include physical machines and/or virtual machines hosted by physical machines. The physical machines may be rackmount servers, desktop computers, or other computing devices. In one embodiment, the server computing devices 110 include virtual machines managed and provided by a cloud provider system. Each virtual machine offered by a cloud service provider may be hosted on a physical machine configured as part of a cloud. Such physical machines are often located in a data center. The cloud provider system and cloud may be provided as an infrastructure as a service (IaaS) layer. One example of such a cloud is Amazon's® Elastic Compute Cloud (EC2®).

The server computing devices 110 may host an MEA interface 150 and one or more virtual environments 155. The MEA interface 150 and virtual environment(s) 155 may be hosted on the same server computing device 110, or may be hosted on separate server computing devices, which may be connected via the network 120.

An MEA 105 (also known as a microelectrode array) is a device that contains multiple plates or shanks through which neural signals are obtained and/or delivered. In embodiments, HD-MEA are used. The plates or shanks are generally arranged in a grid or other array, and serve as neural interfaces that connect neurons 135 to electronic circuitry. The MEA 105 includes a recording chamber 140 that houses many biological neurons 135 and/or a solution or other medium (e.g., a saline solution). These biological neurons 135 may be cultured neurons (e.g., cultured from stem cells) and/or extracted neurons (e.g., extracted from a rat brain). The biological neurons 135 may be from a generic cell line, or may be from a cell line with specific traits to be tested. For example, the biological neurons 135 may be cultured from stem cells of a person having a particular genotype, or from a particular person for whom a test is to be performed, or from a person having a particular pathology. In one embodiment, the neurons 135 comprise cortical cells from embryonic rodent sources. In one embodiment, the neurons 135 comprise cortical cells from human induced pluripotent stem cell (hIPSC) sources.

Neurons can be grown or harvested from numerous sources via multiple methods. Most in-depth in vitro electrophysiological investigations on neural cells have been conducted on primary neurons. This process involves disassociating cortical cells from the dissected cortices of (typically) rodent embryos. These cells are then grown in nutrient rich medium and can be maintained on the order of months. These cultures will develop complicated morphology, with numerous dendritic and axonal connections, leading to functional biological neural networks (BNNs). In some embodiments, such cultures are developed from embryos (e.g., mouse embryos). Properties of monolayers, slices or organotypic cultures can be investigated using a relevant electrophysiological method. The development of spontaneous activity from cultures has been well documented. These developmental stages have also been modelled and found to display emergent connectivity and firing rates that showcase foundational criticality.

As a compelling alternative to use of neuron cultures developed from embryos, advances in stem cell engineering have allowed for stem cells (e.g., induced PSCs, embryonic PSCs, neural precursor cells, etc.) to be efficiently differentiated into monolayers of active cortical neurons which display mature functional properties. This method has the capability of differentiating both upper and lower layer cortical neurons as well as other neural phenotypes. This protocol uses a defined neural induction and maintenance media under specific culture conditions to generate a heterogeneous culture of cortical progenitor cells. Pluripotent cells can be differentiated using variety of techniques, including but not limited to the use of small molecules to recapitulate natural ontogeny, direct reprogramming through the use of viral or other vectors to insert or modify the expression of genes in a cell line to give rise to a specific or varied neural phenotype, or the use of other genetic modification techniques that give rise to a specified or varied neuronal cell types.

In embodiments, neuron cultures (e.g., of long-term cortical neurons and/or other types of neurons) from hIPSCs and/or other sources are implemented to form comparable networks to in vivo neuronal networks within organisms or in vitro networks found in primary neuronal cell cultures, along with appropriate biomarkers showing that cells are not only neural but also more specifically cortical. Along with circumventing ethical issues with harvesting embryonic rodents, hIPSC-derived cells have been demonstrated in embodiments to survive for greater than 6 months with maintained activity and can be grown on an exponential scale, rendering the cost per cell relatively low at high volumes. This allows neuronal ‘wetware’ for computation to be grown and maintained in a functional way.

Historically, neural cultures that have been studied have been sparse neural cultures (e.g., with thousands of neurons) that are two-dimensional. The sparse neural networks have been spread out on a 2D grid such that they do not overlap one another. Such cell arrangements have been used because they are easier to study by enabling individual cells to be studied. However, in some embodiments much denser arrangements of neurons are used than have been used in the past. The dense arrangements of neurons (e.g., with hundreds of thousands to millions of neurons) cause the neurons to overlap one another and form a three-dimensional arrangement in which multiple neurons may be stacked vertically in addition to being arranged on a two-dimensional grid. The dense arrangement of neurons enables the neurons to form spontaneous three-dimensional (3D) structures such as neurospheres, effectively increasing the intelligence of the biological neural network that incorporates the neurons 135. In one embodiment, the dense arrangement of neurons 135 includes at least 10,000 cells per square millimeter, at least 20,000 cells per square millimeter, or at least 50,000 cells per square millimeter. The dense arrangement of neurons enables development of computational assemblies of the neurons 135 in embodiments.

Biological neurons may be placed on an MEA 105 or similar device (referred to herein jointly as an MEA for convenience). The MEA 105 may include electrodes and/or light sources to provide stimulation of neurons. Additionally, or alternatively, the MEA 105 may include chemical generators or emitters that can release chemicals at target locations on the MEA 105. Electrodes may provide electrical stimulation of neurons, light sources may provide optical or light-based stimulation of neurons, and chemical generators or emitters can provide chemical stimulation of neurons. In embodiments, the electrodes, chemical generators/emitters and/or light sources are arranged in a grid. This may enable targeted stimulation of neurons with pinpoint accuracy.

Many light emitting diodes (LEDs) may be arranged in a grid in an embodiment. In another embodiment, a screen may be interposed between one or more light source and the neurons. The screen may be opaque in areas where the neurons are not to be exposed to light, and the screen may be transparent to the light (e.g., may open) at areas where the neurons are to be exposed to light. Which regions of the screen are opaque and which regions are transparent may be adjusted as appropriate. In one embodiment, a display (e.g., a liquid crystal display or organic light emitting diode display) is used as the light source.

In one embodiment, the light sources comprise one or more lasers that may be movable to project light at target coordinates (e.g., at target neurons). For example, the laser may be attached to an actuator or servo-motor that can rotate the laser around multiple axes. In another example, the laser may be fixed, but one or more movable mirrors may direct light from the laser to target neurons or locations.

In embodiments, a grid of chemical emitters is arranged on the MEA. Examples of chemical compounds that may be released by the chemical generators/emitters include neurotransmitters, dopamine, serotonin, glutemate, GABA, ACH, and so on. Neurotransmitters are chemical compounds that condition neurons. For example, neurotransmitters may up regulate or down regulate the internal firing capacity of neurons exposed to those neurotransmitters.

In some embodiments, multiple types of stimulus may be applied to neurons. For example, any combination of electrical, optical and/or chemical stimulus may be applied to neurons sequentially and/or in parallel.

Responsive to certain neurons being excited, those neurons may generate an electrical current, a voltage, a chemical, light, or any combination thereof. This may trigger other nearby neurons to generate an electrical current, a voltage, a chemical and/or light. This process may repeat, where excited neurons then excite still other neurons, and so on.

The MEA 105 may further include one or more optical and/or electrical sensors for detecting neuron activity. Additionally, or alternatively, the MEA 105 may include one or more chemical sensors. In one embodiment, the MEA 105 includes a grid of electrodes that can measure voltage and/or current at locations of neurons. In embodiments, the same grid of electrodes can be used both for excitation of neurons and for measuring electrical activity of neurons responsive to such excitation. In one embodiment, a grid of chemical sensors is arranged on the MEA 105 to detect locations at which particular chemicals are present.

Neurons can be designed to fluoresce under certain conditions (e.g., responsive to stimulus). In such instances, optical sensors may be used to detect locations on the MEA at which neurons are fluorescing (e.g., to detect which neurons have been stimulated and are generating an output). In one embodiment, the MEA 105 includes a grid of optical sensors. In one embodiment, the MEA 105 includes one or more cameras. Different regions within the fields of view of the cameras may be associated with different neurons and/or MEA coordinates. Images generated by the camera(s) can be used to determine locations on the MEA at which neurons have been activated. For example, each pixel in an image may be associated with a particular x, y location on MEA 105. The camera can generate an image which can be analyzed to determine which x, y locations on the MEA 105 have neurons that fluoresced at a given time.

One or more of the MEA(s) 105 may be an active MEA that includes an integrated circuit 145 (or multiple integrated circuits), such as a CMOS circuit. The integrated circuit(s) 145 may include processing logic (e.g., a general purpose or special purpose processor), a network adapter, a digital to analog converter (DAC), an analog to digital converter (ADC), and/or other components. The network adapter may be a wired network adapter (e.g., an Ethernet network adapter) or a wireless network adapter (e.g., a Wi-Fi network adapter), and may enable the MEA(s) 105 to connect to network 120. In one embodiment, the integrated circuit 145 includes a processing device, which may be a general purpose processor, a microcontroller, a digital signal processor (DSP), a programmable logic controller (PLC), a microprocessor or programmable logic device such as a field programmable gate array (FPGA) or a complex programmable logic device (CPLD). In one embodiment, the integrated circuit 145 includes a memory, which may be a non-volatile memory (e.g., RAM) and/or a volatile memory (e.g., ROM, Flash, etc.). In one embodiment, the integrated circuit 145 is a system on a chip (SoC) that includes the processing device, memory, network adapter, DAC, and/or ADC.

In one embodiment, one or more of the MEA(s) 105 is a passive MEA that is connected to one or more integrated circuits 145 via one or more leads and/or a printed circuit board (PCB).

In one embodiment, one or more of the MEAs 105 further includes an optical source that is capable of providing optical impulses to specified 2D coordinates in the 2D grid. The optical source may include light emitting elements (e.g., light emitting diodes (LEDs), light bulbs, lasers, etc.) that are capable of emitting light having one or more specified wavelengths. Accordingly, optogenics may be used to manipulate neural activity. Additionally, lasers of specific wavelengths may be used for highly accurate targeting of specific neurons. The response to optical stimulation may then be measured by the electrodes in the MEA(s) 105. Unlike electrical stimulation, light stimulation manipulates specific cells (e.g., neurons) that may express a targeted opsin protein, thereby making it possible to investigate the role of a subpopulation of neurons in a neural circuit. In some embodiments, immunofluorescence of specifically a modified calcium that gets cleaved and activated when they enter the neurons can also be paired with a camera to image activation of neurons. Accordingly, in embodiments the MEA 105 provides optical stimulation to specified 2D coordinates and measures electrical signals generated by neurons 135 in response.

In one embodiment, one or more of the MEAs 105 provide electrical stimulation to specified 2D coordinates in the 2D grid, but optical signals are measured. MEAs 105 may include one or more optical sensors capable of optically detecting electrical excitation of neurons and generating optical signals based on such detected electrical excitation of the neurons. Accordingly, optogenics may be used to detect neural activity. The optical sensors may include charge coupled devices (CCDs), complementary metal oxide (CMOS) devices, and/or other types of optical sensors.

Mechanisms for optically detecting neural activity are discussed in greater detail below. In some embodiments, immunofluorescence of specifically modified calcium that get cleaved and activated when they enter the neurons can be paired with one or more image sensors to image activation of neurons. In some embodiments genetically encoded voltage detectors may be introduced into cells at a given point and used to detect activation of neurons when stimulated with light. In some embodiments luciferase based reactions may be introduced into the cells and paired with another method of detecting voltage changes in neurons to detect changes in voltage without the need for external light stimulation.

In one embodiment, a fully optical system may be used instead of an MEA. In such an embodiment, a substrate on which the neurons are plated and/or additional components may include an optical source that is capable of providing optical impulses to specified 2D coordinates in a 2D grid. The optical source may include light emitting elements (e.g., light emitting diodes (LEDs), light bulbs, lasers, etc.) that are capable of emitting light having one or more specified wavelengths. Additionally, lasers of specific wavelengths may be used for highly accurate targeting of specific neurons. Additionally, the substrate and/or other components may include one or more optical sensors capable of optically detecting electrical excitation of neurons and generating optical signals based on such detected electrical excitation of the neurons. Accordingly, optogenics may be used to manipulate and detect neural activity.

In the case of an active MEA 105, on-chip signal multiplexing may be used to provide a large number of electrodes to achieve a high spatio-temporal resolution in recording of electrical and/or optical signals and providing of electrical impulses (e.g., as with an HD-MEA). Moreover, weak neuronal signals can be conditioned right at the electrodes by dedicated circuitry units, which provide a large signal-to-noise ratio. Finally, analog-to-digital conversion may performed on chip, so that stable, digital signals are generated.

Biological neurons can be designed to fluoresce, generate a current, generate a voltage, release a chemical compound, and so on via various mechanisms. In some embodiments, excitation of the biological neurons stimulates changes in cell membrane characteristics, which can cause them fluoresce, generate a current, generate a voltage, release a chemical compound, and so on. In some embodiments, ion channels, proteins, intra-membrane structures, extra membrane structures, and/or transmembrane structures generate a current, voltage, light and/or a chemical compound responsive to stimulation of a neuron. In some embodiments, channels (e.g., ion channels) are opened and/or closed (e.g., responsive to exposure to light, to a voltage, to a current, to a chemical compound, etc.) in cell membranes to generate a current. In some embodiments, neurons may be designed to directly generate a voltage (e.g., via a protein).

In some embodiments, biological neurons create ion currents through their membranes when excited, causing a change in voltage between the inside and the outside of the cell. When recording, the electrodes on an MEA transduce the change in voltage from the environment carried by ions into currents carried by electrons (electronic currents). When stimulating, electrodes may transduce electronic currents into ionic currents through the MEA. This triggers the voltage-gated ion channels on the membranes of the excitable neurons, causing the neuron to depolarize and trigger an action potential.

In some embodiments, neurons express a reporter (e.g., a gene reporter) responsive to stimulation. The expressed reporter may cause the neurons to fluoresce at a certain wavelength and/or to release a chemical compound. For example, neurons may be designed to have a fluorescent protein that fluoresces when stimulated. In another example, neurons may be designed to cleave to release another protein or chemical when stimulated (e.g., when stimulated via light). In some embodiments, light can be used to target an organelle of a neuron cell. In some embodiments, light can trigger a reaction in, on or through a cell membrane of a neuron cell. In some embodiments, stimulation of a neuron cell can open or close ion channels, activate, inflate, inhibit, or cleave a protein in the neuron cell, and so on.

In one embodiment, a protocol works via inhibiting the dual SMAD signaling pathway of neuron cells. SMADs comprise a family of structurally similar proteins that are the main signal transducers for receptors of the transforming growth factor beta (TGF-B) superfamily, which are important for regulating cell development and growth. The abbreviation refers to the homologies to the Caenorhabditis elegans SMA (“small” worm phenotype) and MAD family (“Mothers Against Decapentaplegic”) of genes in Drosophila. SMAD inhibition has been found to drive differentiation toward the anterior neuroectodermal lineage.

The size and shape of a recorded signal may depend upon the nature of the medium (e.g., solution) in which the neuron or neurons are located (e.g. the medium's electrical conductivity, capacitance, and homogeneity), the nature of contact between the neurons and the electrodes (e.g. area of contact and tightness), the nature of the electrodes (e.g. its geometry, impedance, and noise), the analog signal processing (e.g. the system's gain, bandwidth, and behavior outside of cutoff frequencies), and data sampling properties (e.g. sampling rate and digital signal processing). For the recording of a single neuron that partially covers a planar electrode, the voltage at the contact pad is approximately equal to the voltage of the overlapping region of the neuron and electrode multiplied by the ratio the surface area of the overlapping region to the area of the entire electrode. An alternative means of predicting neuron-electrode behavior is by modeling the system using a geometry-based finite element analysis in an attempt to circumvent the limitations of oversimplifying the system in a lumped circuit element diagram.

In some embodiments, blinding is used to facilitate an ability to distinguish between detection of electrical signals, chemical signals and/or optical signals generated by neurons and electrical signals, chemical signals and/or optical signals generated by the MEA 105 based on instructions. Blinding prevents the stimulation of electrodes/optical sensors/chemical sensors caused based on instructions from the MEA interface 150 and/or virtual environment 155 from interfering with detection of electrical signals/light/chemical signals generated by neurons 135. One or more blinding schemes may be used. The MEA interface 150 may apply a blinding technique to determine a first subset of signals to process and a second subset of signals to ignore, delete, or filter out.

In some embodiments, MEA interface 150 and/or integrated circuits 145 may know when electrodes are stimulated and/or which electrodes are stimulated. The electrical fields generated by stimulating electrodes may be much larger than the electrical fields generated by neurons 135. Accordingly, in one embodiment, detected electrical signals are applied to a filter, which may filter out electrical fields/signals that are greater than a threshold size (e.g., that are detected by more than a threshold number of electrodes), where these electrical fields/signals are caused by active stimulation of electrodes by the integrated circuit 145 and/or MEA interface 150. Such filtering may be performed, for example, by integrated circuit 145 and/or server computing device 110. However, smaller electrical fields caused by neurons 135 may only be detected by a small number of electrodes, and may thus not be filtered out. Additionally, signals may be filtered out based on voltage. For example, electrical signals caused by electrodes 130 may have much larger voltages than electrical signals generated by neurons 135. For example, electrical signals generated by electrodes 130 may have voltages on the order of a thousandth of a volt, and electrical signals generated by neurons 135 may have voltages on the order of a millionth of a volt. Thus, electrical signals may additionally or alternatively be filtered based on voltage. Similar blinding techniques may also be applied for optical and/or chemical signals for the above and below described blinding techniques.

In one embodiment, a rough timing of when electrical signals are output to electrodes and/or optical signals are output via optical components is known. Knowledge of timing may not be perfect because of unpredictable delays in command delivery. Accordingly, blinding may be performed by ignoring electrical and/or optical signals output at or around the time that electrical and/or optical signals are output to the electrodes 130 and/or optical components. In one embodiment, an internal counter of commands is maintained (e.g., by server computing device 110 and/or integrated circuit 145). Each time the internal counter increments, this may indicate that new electrical and/or optical signals are output to one or more electrodes. Accordingly, in one embodiment when the internal counter increments, electrical and/or optical signals are ignored for a set amount of time.

In some embodiments, multiple blinding techniques may be combined.

In one embodiment, a blinding method (e.g., consensus blind) based on blinding all signals when >15 simultaneous large (>75 mV) spikes were detected, is implemented to block stimulation delivered by the system from being registered as cellular activity. In some embodiments, a new blinding method is implemented, which is termed ‘command count blinding’. This method blinds a readout of all motor activity when a command was sent to generate any form of stimulation. During testing this was found to be significantly more robust than the previously used consensus blinding and enabled increased density and variability of sensory stimulation.

The MEA(s) 105 can be used to perform electrophysiological experiments on dissociated cell cultures (e.g., cultures of biological neurons). With dissociated neuronal cultures, the neurons spontaneously form biological neural networks. This phenomena may be increased by using very dense neural cultures, as set forth above. The MEA(s) 105 may include an array of electrodes 130 and the recording chamber 140 that contains a living culture of biological neurons 135 in a nutrient rich solution that will keep the biological neurons alive. The array of electrodes 130 may be a planar array (e.g., a two-dimensional (2D) grid) or a three-dimensional (3D) array (e.g., a 3D matrix). The array of electrodes 130 that may be used to take measurements at 2D coordinates (or 3D coordinates) at high spatial and temporal resolution at excellent signal quality. Additionally, the array of electrodes 130 may be used to apply electrical impulses at the 2D coordinates or 3D coordinates.

By plating cortical neurons on one or more HD-MEA (e.g., MaxOne MEA by Maxwell Biosystems) in embodiments, mapping of the in-vitro development of electrophysiological activity in neural systems at high spatial and temporal resolution was achieved. In one embodiment, robust activity in primary cortical cells from E15.5 rodents was found at DIV 14 where bursts of synchronized activity was regularly observed. In contrast, in one embodiment similar synchronized bursting activity was not observed in cortical cells from an iPSC background until DIV 73. In one example, single spiking neurons were identified in these latter cultures as early as DIV 42 in an embodiment, however more ordered clustered spiking was not observed until approximately DIV 82.

Along with recording changes in electrical activity brought about from action potentials, the MEA 105 has the potential to stimulate cells at a range of voltages. Providing external electrical stimulation is relatively non-invasive to cells, and effectively elicits action potentials or responses in a comparable manner to internal electrical stimulation. With an appropriate coding scheme, external electrical stimulations are able to convey a range of information. Different coding schemes are discussed in greater detail below. Through this method there is the capacity to not only ‘read’ information from a neural culture, but to ‘write’ data into one.

The MEA interface 150 may be responsible for translating between inputs/outputs of the virtual environment(s) 155 and the inputs/outputs of the MEA(s) 105. The inherent property of neurons to have a shared ‘language’ of electrical activity between each other means links between silicon and biological systems can be formed through electrical stimulation. For this reason, electrical stimulation (as well as optical and chemical stimulation) may be used to induce neuronal plasticity in vitro or to provide structured information that cells to facilitate embodiment of these cells in an environment.

Embodiments provide a neural interface (referred to as MEA interface 150) for interfacing a biological neural network (e.g., a neuron culture) with an electronic system (e.g., with a virtual environment or logic executing on a computing device). The role of the neural interface is to perform encoding, which includes arranging information output by processing logic into a format that can be delivered to the biological neural network and understood by the biological neural network, and decoding, which includes arranging information output by the biological neural network into a format that can be delivered to and understood by processing logic. The neural link performs the core function of taking disordered electrical, chemical and/or optical signals from hundreds of electrodes, and then interpreting those disordered electrical, chemical and/or optical signals and doing something useful based on those disordered electrical, chemical and/or optical signals. For example, a neural interface as described herein may be used to enable a biological neural network to control activity of a robot (e.g., a robot arm), to play a game, to interact with a virtual environment, to drive an automobile, and so on. While embodiments herein discuss the biological neural network being a neuron culture of neurons 135 on an MEA 105 (which may be a traditional MEA or an optical or electrical/optical analog), in alternative embodiments the biological neural network may be a part of a brain of a living person or animal. The neural interface described in embodiments herein may be used as a bridge between neurons of a human brain and processing logic and/or a computing device.

In one embodiment, the neural interface (e.g., MEA interface 150) provides a vectorized bridge that can convert temporal/rate encoding and/or place/position encoding into vectors and/or tensors and that can convert vectors and/or tensors into temporal/rate encoding and/or place/position encoding. To effectively interface digital systems with biological neural activity, an effective mechanism for taking real time in-vitro neural activity and translating that neural activity into vectors and/or tensors (e.g., lists of numbers) can be important. Vectors and tensors are static lists of values, and the neural interface converts these static lists of values into actual potential spiking of electrodes, where the potential spiking can be performed according to rate-based coding, place-based coding and/or mixed coding schemes. Accordingly, the potential spiking (e.g., stimulus patterns) can be performed according to rate coding and also in terms of a 2D or 3D spatial layout. Additionally, the neural interface (e.g., MEA interface 150) converts measured potential spiking (e.g., electrode signals over time and/or space) into static lists of values (e.g., vectors and/or tensors). The neural interface provides a biologically compatible mechanism for choosing when and where to provide stimulation, for example. This can include determining which electrodes to apply electrical signals to (or which light sources to apply signals to for generation of optical signals), voltages to use, a current to use, a frequency to use, and so on. Thus, a vector or tensor may include a set of values that capture voltage levels in time and by electrode, for example. While the neural interface is shown to be on server computing device 110 in some embodiments, the neural interface may also be on MEAs (e.g., implemented using integrated circuits 145) in embodiments. In some embodiments, some operations of the neural interface are performed by MEA 105 and other operations of the neural interface are performed at the server computing device 110 (e.g., by MEA interface 150).

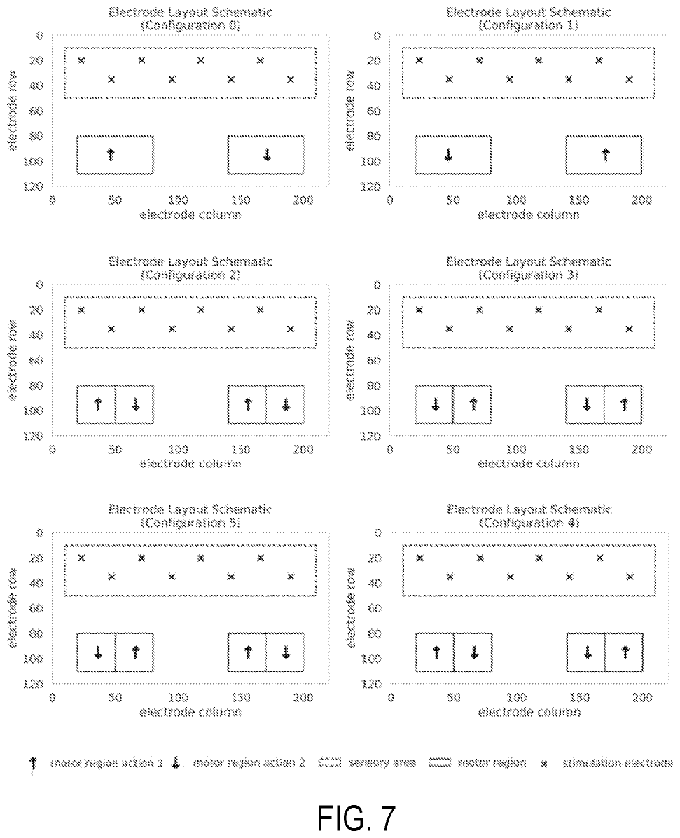

To train a neural culture to perform tasks (i.e., to play Pong or perform other tasks), an area of an MEA (e.g., grid of electrodes) or other device (e.g., a fully optical device) may be divided into regions, and each region may be assigned a role. An example is set forth below with reference to FIG. 7. One role is a simulated sensory area, in which inputs associated with a virtual environment are provided to the neural culture. The simulated sensory area may also be used for feedback (e.g., punishment stimulus and/or reward stimulus) during training of the neural culture. Alternatively a role of feedback may be assigned to another region. Other roles may also be assigned for each type of output that the neural culture will be trained to produce. For example, in the case of training the neural culture to play the game Pong, there may be one role for moving a paddle up (or left) and one role for moving the paddle down (or right). The role of moving the paddle up may be assigned to one region, and the role of moving the paddle down may be assigned to another region. The number of possible roles may be arbitrarily large and the options for what they represent are nearly limitless.

As set forth above, place-based or position-based encoding schemes may be used in embodiments, in which a different meaning is associated with stimulation (e.g., electrical, chemical and/or optical stimulation) in different locations on the MEA. For example, stimulation in the sensory area may represent a state of a virtual environment. Encoding of stimulation in the sensory area may be based on a correlation between position of electrodes and information from the virtual environment (place-based encoding). In one example, place based encoding is used to convey a distance between a paddle and a ball on a first axis (e.g., x-axis) and/or on a second axis (e.g., y-axis). Thus, which electrodes output electrical impulses may indicate where the ball is on the x-axis and/or where the ball is on the y-axis in an example. Similarly, for decoding where neural activity is detected may indicate what action to decode the neural activity into. For example, firing of neurons in a first region may indicate that the paddle is to move one direction and firing of neurons in a second region may indicate that the paddle is to move another direction.

In some embodiments, rate-based encoding schemes may also be used. For rate-based encoding schemes, the frequency of neuron activation may be associated with a meaning. For example, a first frequency of neuron activation may convey a first meaning (e.g., paddle is near ball in virtual environment, or a first distance between the ball and the paddle in a first axis) and a second frequency of neuron activation may convey a second meaning (e.g., paddle is far from ball in virtual environment, or a second distance between the ball and the paddle in the firs axis). Similarly, for decoding the frequency at which neural activity is detected may indicate what action to decode the neural activity into. For example, firing of neurons in a at a first rate may indicate a first speed to move the paddle and firing of neurons at a second rate may indicate a second speed to move the paddle.

In one example, a constant unpredictable stimulus may be provided to the culture through one DAC at a varied frequency while a simultaneous and secondary place and rate coded stimulus is provided through a secondary DAC. The combination of these signals could provide not only a vectorized direction to a target in a multidimensional space but also inform the variance away from the given target.

In further embodiments, a mixed encoding scheme may be used, in which some information is conveyed based on position of electrical, chemical and/or optical signals provided to neurons, and other information is conveyed based on frequency of electrical, chemical and/or optical signals provided to the neurons. Encoding of stimulation in the sensory area may be based on a correlation between position of electrodes and information from the virtual environment (place-based encoding) and/or based on a correlation between a frequency of firing of electrodes (rate-based encoding). In one example, in which a mixed encoding scheme is used to convey information about the virtual environment to the neurons, place based encoding is used to convey a distance between a paddle and a ball on a first axis (e.g., x-axis) and rate-based encoding is used to convey distance between the paddle and the ball on a second axis (e.g., y-axis). Thus, which electrodes output electrical impulses may indicate where the ball is on the x-axis and the frequency that those electrodes generate impulses may indicate where the ball is on the y-axis in an example. In some embodiments, a mixed decoding scheme may be used, in which some information is conveyed based on position of electrical, chemical and/or optical signals output by neurons, and other information is conveyed based on frequency of electrical, chemical and/or optical signals output by the neurons.

In some embodiments, a higher density of informational input and predictable stimulus yields improved performance. In some embodiments, stimulation is delivered in the theta range only (4 Hz). This is justified as theta rhythms has been proposed to be linked to the active intake of sensory stimuli and stimuli sampling. However, compelling research in animal models suggests that beta frequency (approx. 15-40 Hz) rhythms may be involved in top-down processing to promote feedback interactions across the visual area. Beta oscillations have also been linked to anticipation of visual stimuli and the subsequent cueing of a visual response. Accordingly, in embodiments stimulation is delivered at the beta frequency range.

For some embodiments, standard static purely place-coded data may not be ideal, as it is difficult to code for more than a single type of information with only place based coding. A single fixed frequency stimulation in general may only code for a single dimension. Using a variable frequency grants the ability to convey additional information, such as the ability to communicate the relative distance from the paddle on the other axis for the Pong example. Given this, it was deemed desirable to investigate the effect of using a combination rate and place coded signal. In some embodiments, stimulus activity changes between 4-40 Hz based on conditions within the virtual environment (e.g., based on the distance to the paddle on the x-axis in the Pong example). In some embodiments, the electrodes at which stimulus activity is provided additionally or alternatively changes based on conditions within the virtual environment according to place coded information (e.g., place coded information may communicate distance from the paddle on the y-axis in the Pong example).

As mentioned above, rate based and/or position based decoding schemes may also be used for decoding the signals output by the neurons. For example, place-based decoding schemes may be used to interpret signals from a first output region (e.g., first motor region) as a first action command and to interpret signals from a second output region (e.g., second motor region) as a second action command. In another example, rate-based decoding schemes may be used to interpret signals from one or more output regions, including a continuum of motor regions. For example, a first rate of signals may indicate to move a paddle left, and a second rate of signals may indicate to move the paddle right.

In embodiments, each place coded region may represent values to an x-axis while rate coting may represent values to a y-axis. An example of a mixed place-rate code could be a virtual agent like a mouse which uses virtual whiskers to navigate its environment. As each whisker is at a fixed spatial location and the distance that it is touching a specific object is translated to a rate coded pattern, having different whiskers at their specific locations with differing rates of action potentials allows for a “3D sensing” of its surrounding environment.

In a furtherance of the Pong example, a first frequency of signals in a first motor region may indicate both that a paddle should move in a first direction and a velocity to move that paddle in the first direction. A second frequency of signals in the first motor region may indicate both that the paddle should move in the first direction and second velocity to move that paddle in the first direction. A first frequency of signals in a second motor region may indicate both that the paddle should move in a second direction and a first velocity to move that paddle in the second direction. A second frequency of signals in the second motor region may indicate both that the paddle should move in the second direction and a second velocity to move that paddle in the second direction. Mixed coding and/or decoding, optionally including rate-based coding and place-based coding, may be used to convey many other meanings, depending on the virtual environment and the task or tasks that a neural culture is trained to perform.

Coding and/or decoding schemes may also be at least in part based on voltage levels and/or current levels. Accordingly, a mixed coding scheme may convey information based on position, rate, voltage and/or current for encoding and/or decoding of information.

Research has traditionally used open-loop systems where the stimulus is divorced from the resulting neural activity. This work has been limited to demonstrating that electrical stimulation can induce long-lasting responses in cultures of neural cells but have been unable to guide these responses in a way to elicit or observe meaningful goal-directed behavior. These studies have enabled a degree of understanding into the mechanisms through which cells self-organize. In contrast, in embodiments closed-loop adaptive training algorithms may be used for in vitro neural networks to modulate firing patterns and activity states; and are significantly more effective at altering neuroelectric activity than open-loop stimulation patterns. Closed-loop systems afford an in vitro culture embodiment by providing feedback on the causal effect of the behavior from the neural culture.

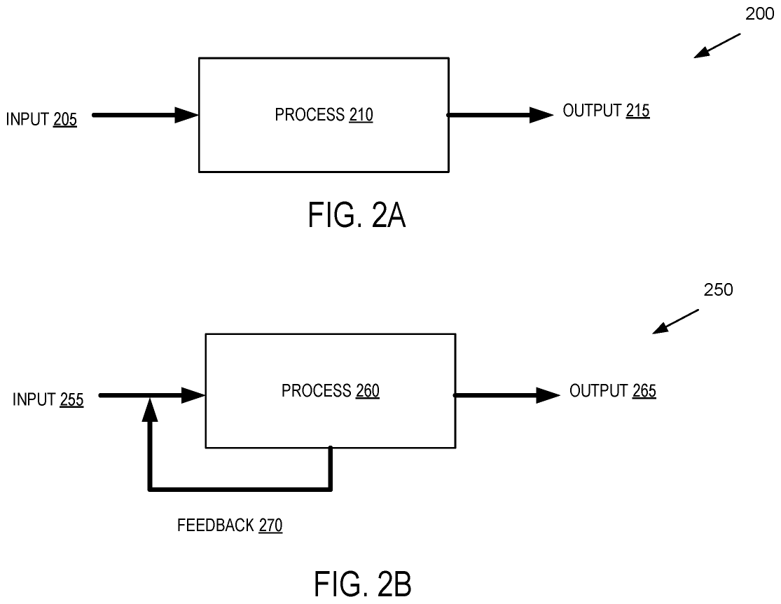

FIGS. 2A-B illustrate the difference between an open-loop feedback system and a closed-loop feedback system. FIG. 2A illustrates an example open-loop feedback system 200. An open-loop feedback system (also referred to as a non-feedback system or open-loop system) is a continuous control system in which an output 215 generated by some process 210 has no influence or effect on a control action or an input 205. Accordingly, in an open-loop feedback system 200, an input 205 is processed by a process 210 to generate an output 215. Later, a new input that is not influenced by the output 215 may be input into the process 210 to generate a new output. Therefore, an open-loop system is expected to act on its input 205 or set point regardless of the final result (output 215).

FIG. 2B illustrates a closed-loop feedback system 250 (also referred to as a closed-loop system). Similar to an open-loop system, for a closed-loop system an input 255 is provided to a process 260, which operates on the input 255 to generate an output 265. Accordingly, a closed-loop system uses a similar forward path as an open-loop system. However, a closed-loop system 250 has one or more feedback loops between its output 265 and input 255 that provide feedback 270 that can be used as further input into the process 260. A closed-loop system may be used that enables neurons to modify themselves in order to affect a virtual or physical environment and that enables the environment to modify how the neurons behave. Closed-loop systems may be designed to automatically achieve and maintain a target output condition by comparing it with an actual condition. In some embodiments, the closed-loop system generates an error signal which is the difference between the output 265 and a reference value or state. In other words, a “closed-loop system” is a fully automatic control system in which its control action is dependent on the output in some way.

Demonstrated embodiments have shown that closed-loop feedback system (e.g., an electrophysiological closed-loop feedback system) results in significant network plasticity and potentially behavioral adaption over and beyond what can be achieved with open-feedback systems. It is believed that providing feedback to the system about the result of the state the system adopts provides the required information for neural systems to adapt and alter behavior as required for a given aim. A closed-loop feedback system such as an electrophysiological closed-loop system functions by taking a given information generated directly or indirectly from the function of a biological system or systems, having this information—or a derivative of this information—be applied or communicated to an external environment or system thereby altering and/or impacting the external environment, then applying or communicating the changed environmental state to the biological system or systems. In one example this could involve providing electrical stimulation to biological neurons, recording the activity of the biological neurons, then using the recorded activity as a metric to control an action of a simulated or physical device. The outcomes of this control on the simulated or physical device are then relayed to the biological neurons via changes in the electrical stimulation provided.

Returning to FIG. 1, each virtual environment 155 (or experiment logic or real environment) may include some processing logic that may receive inputs from one or more MEAs 105 and/or an external source, and that may generate outputs. One example of processing logic of a virtual environment is a machine learning model such as an artificial neural network, deep neural network, convolutional neural network, recurrent neural network, etc. Other machine learning models included in virtual environments may apply a k-nearest neighbors algorithm, a learning vector quantization, a self-organizing map, regression analysis, a regularization algorithm, and so on. Another example of processing logic of a virtual environment is an application executing on the server computing device. For example, the application may be a game (e.g., Pong), and the biological neurons 135 on the MEA 105 may be trained to play the game. The application may also be any other program that includes one or more tasks to be performed or problems to be solved, and the biological neurons 135 on the MEA 105 may be trained to perform the task or tasks.

The server computing device 110 may provide one or more application programming interfaces (APIs) that enable third parties to upload virtual environments 155 and connect those virtual environments 155 to one or more MEAs 105. Each virtual environment 155 may be assigned one or more MEAs 105, and may train the neurons 135 on those MEAs 105 to perform some task, as discussed in greater detail below. Each virtual environment 155 may be assigned a virtual environment identifier (ID), and each MEA 105 may be assigned an MEA ID. Virtual environment IDs may be associated with MEA IDs 105 in a database or other data store, which may be maintained by the server computing device(s) 110. In embodiments, one or more virtual environments may be virtualizations of real environments. Such virtual environments may be updated in real time or near-real time based on a state of the real environment reflected by the virtual environment. This enables the virtual environment to interface with the biological neurons and also to provide instructions to control real world systems.

Once a virtual environment 155 is paired with an MEA 105, that virtual environment may begin providing digital input signals for the MEA 105. The virtual environment 155 may generate a digital input signal, which may be, for example, a vector (e.g., a sparse vector and/or floating point vector), a message complying with some communication protocol, or a 2D or 3D matrix of values. The MEA interface 150 may include information on the array of electrodes 130 of the MEA 105, light sources, chemical emitters, optical sensors, chemical sensors, and so on. This may include information on the number of electrodes 130 and how the electrodes 130 are arranged in the recording chamber 140 (e.g., for a 2D grid of electrodes, the number or rows and columns of electrodes), for example. The MEA interface 150 may convert the digital input signal from the virtual environment 155 into instructions for one or more electrical, chemical and/or optical impulses according to an encoding scheme, where each electrical, chemical and/or optical impulse instruction is associated with a 2D coordinate or a 3D coordinate. Each electrical, chemical and/or optical impulse instruction may further include information on an amplitude or intensity of the impulse to apply, a frequency or wavelength of the impulse to apply, timing of when to apply the electrical, chemical and/or optical impulse and/or a current of the impulse to apply. Accordingly, the information for each impulse may be a tuple that includes one or more of (x coordinate, y coordinate, z coordinate, intensity/amplitude, frequency/wavelength, current, or other coded information).

Once the MEA interface 150 converts the digital input signal from the virtual environment into information for one or more optical, chemical and/or electrical impulses (referred to herein as encoding), it may send the information to the appropriate MEA 105. As discussed above, such encoding may be performed according to an encoding scheme, which may be a place-based encoding scheme, a rate-based encoding scheme, a hybrid encoding scheme, or another type of encoding scheme. An integrated circuit 145 of the MEA 105 or computing device may convert the information into one or more analog signals for the optical or electrical impulses (e.g., using a DAC). The MEA 105 applies the one or more analog signals to appropriate electrodes 130 (or light emitting elements or chemical emitters) to apply the optical, chemical and/or electrical impulses at the specified coordinates and/or with the specified intensity/amplitude, frequency/wavelength, and so on.

In some embodiments, one or more cameras are used to measure activated neurons. In embodiments, neurons may be modified to fluoresce when they fire, and the fluorescence may be captured by image sensors (e.g., cameras). In one embodiment, modified calcium sensors may be used to cause the neurons to fluoresce when cell calcium levels change. The calcium sensor may be cleaved and activated when it enters a by cell esterases may allow neuron (which may happen when a neuron or pair of neurons is activated). The cleaving of the calcium sensor it it to exhibit fluorescence in response to binding cell calcium.

In one embodiment, genetically encoded voltage indicators (GEVI) are used. GEVIs are fluorescent protein reporters of membrane potential. A GEVI is therefore a protein that can sense membrane potential in a cell and relate the change in voltage to a form of output. In embodiments, the protein generates the output by fluorescing. A GEVI can have many configuration designs in order to realize voltage sensing function. In embodiments, the GEVI is on or in the cell membrane. The GEVI senses a voltage difference as part of a voltage-sensitive domain (VSD)-based sensor to report the voltage difference by a change in fluorescence. In another embodiment the GEVI can be a rhodopsin (a G-protein coupled receptor found in rod cells in the retina) based sensor. In another embodiment the GEVI could be a rhodopsin-fluorescence resonance energy transfer (FRET) sensor.

In one embodiment, a Bioluminescence resonance energy transfer (BRET) is used. In one embodiment, BRET is based on the energy derived from a luciferase reaction that can be used to excite a fluorescent protein if the fluorescent protein is near the luciferase enzyme. A BRET includes a fusion of donor (luciferase) and acceptor (fluorescent) molecules to proteins of interest. Energy is transferred through non-radiative dipole-dipole coupling from the donor to the acceptor when in proximity, resulting in fluorescence emission at a specific wavelength. The energy emitted by the acceptor relative to that emitted by the donor is termed the BRET signal. It is dependent upon the spectral properties, ratio, distance and relative orientation of the donor and acceptor molecules as well as the strength and stability of the interaction between the proteins of interest.

In one embodiment GEVIs are expressed alongside Bioluminescence resonance energy transfer (BRET) techniques to enable emission (e.g., a luciferase based emission) of light when voltage changes occur in a cell. This enables observation of firing cells without additional fluorescence imaging. In one embodiment, the plurality of in vitro biological neurons in the MEA each comprise a genetically encoded voltage indicator (GEVI) and a bioluminescence resonance energy transfer (BRET) that fluoresce to generate the second signals. In another embodiment through genetic and protein engineering either a rhodopsin based voltage sensitive unit or another VSD acts to cleave or transfer a bound luciferase from a unit with an embedded fluorophore when voltage changes, thereby emitting a wavelength of light that marks a voltage change—such as an action potential—inside or around a cell. In another embodiment the change in membrane voltage will influence the local electrochemical potential triggering the release of energy bound in the luciferase and thereby exciting the fluorophore.

The one or more cameras can detect the fluorescence and determine a location that the fluorescence occurred. Alternatively, the MEA or computing device can receive the image from the camera and determine where the fluorescence occurred. In particular, the MEA or computing device may determine coordinates of where light was measured from the image. The MEA or computing device may then generate a digital representation of the locations at which light was detected (e.g., locations that exhibited immunofluorescence).

The MEA interface 150 may receive the digital representation from the MEA 105. The MEA interface 150 may then generate a response message based on the digital representation received from the MEA 105 (e.g., perform decoding). As discussed above, such decoding may be performed according to a decoding scheme, which may be a place-based coding scheme, a rate-based coding scheme, a hybrid coding scheme, or another type of coding scheme. Generating the response message may include converting the representation into a format that is readable by the virtual environment 155. This may include converting the representation (e.g., which may be in the form of a matrix of values representing electrical signals at various coordinates) into a sparse vector or tensor in one embodiment. The MEA interface 150 may then send the response message to the virtual environment 155.

The virtual environment 155 may process the response message, and based on the processing may determine whether the electrical signals output by the neurons 135 correspond to a target set by the virtual environment 155 (or other logic). The target may be unknown to the MEA interface 150 and/or MEA 105. If the electrical signals corresponded to the target, then the virtual environment 155 may use an API of the MEA interface 150 to send a positive reinforcement training signal to the MEA interface 150. The positive reinforcement training signal indicates that signals (e.g., electrical, chemical and/or optical signals) output by the neurons 135 in response to the digital input signal satisfied some criterion of the virtual environment 155 (e.g., indicates that some target objective of the virtual environment was satisfied by the representation of the one or more electrical signals). Alternatively, in some embodiments no positive reinforcement training signal is generated or sent to the MEA interface. Also, if the signals fail to corresponded to the target, then the virtual environment 155 may use the API of the MEA interface 150 to send a negative reinforcement training signal to the MEA interface 150. The negative reinforcement training signal indicates that signals output by the neurons 135 in response to the digital input signal failed to satisfy some criterion of the virtual environment 155 (e.g., indicates that some target objective of the virtual environment was not satisfied by the representation of the one or more electrical signals). Alternatively, in some embodiments no negative reinforcement training signal is generated or sent. Instead, all inputs to the neurons 135 may be paused for a brief time period if the signals fail to correspond to the target. In one embodiment, positive reinforcement signals are used, but negative reinforcement signals are not used. In one embodiment, both positive and negative reinforcement signals are used. In one embodiment, negative reinforcement signals but not positive reinforcement signals are used.

In one embodiment, positive reinforcement signals are or include predictable signals, while negative reinforcement signals are or include unpredictable signals. A predictable signal may be a signal that follows a set pattern. In theory, neurons (and the brain) are prediction machines, and act in a manner to cause predictable stimuli (e.g., desire predictable stimuli). Accordingly, unpredictable stimuli may be used as a form of punishment, and predictable stimuli may be used as a form of reward, whether the predictable or unpredictable stimuli is electrical stimuli, optical stimuli, or chemical stimuli. Predictable and unpredictable stimuli may be used in embodiments to shape the behavior of neurons. In an example, an MEA may include multiple sensory electrodes (also referred to as stimulus electrodes), such as 2-20 (e.g., 8 or 10) sensory electrodes or a continuum of sensory areas over a given predefined area. These sensory electrodes (and/or optical components) may deliver electrical and/or optical signals according to one or more rules of a virtual environment 155 and/or training logic Similarly, chemical emitters may deliver chemical signals according to one or more rules of a virtual environment 155 and/or training logic Similarly, optical components (e.g., light sources) may deliver optical signals according to the one or more rules of the virtual environment 155 and/or training logic. This can train the neurons to expect these sensory electrodes (or some subset of the sensory electrodes) to receive electrical stimulation under certain predictable circumstances according to the rules of the virtual environment 155. Similarly, this can train the neurons to expect optical and/or chemical simulation under certain predictable circumstances according to the rules of the virtual environment. When such electrical, chemical and/or optical signals are received as expected, this acts as a reward to the neurons. However, electrical, chemical and/or optical signals may be delivered in a random manner or according to some other rule or rules that have not been applied for the virtual environment 155, which are all unpredictable stimuli.

In an example, if the virtual environment is the game Pong, then one or more of the sensory electrodes that are associated with a location in proximity with a moving ball may be excited when the neurons cause a paddle to be moved in front of the moving ball, where the excitation of these sensory electrodes would be a predicable stimulus. However, if the paddle in the virtual environment 155 is not moved in front of the ball, then all or a random sampling of the sensory electrodes may be excited, where the excitation of these sensory electrodes would be an unpredictable stimulus.

An unpredictable stimulus may be, for example, a random sequence of electrical, chemical and/or optical signals by a random selection of sensory electrodes, where the random sequence does not have any structure. Experimentation has shown that unpredictable stimuli may disrupt the internal dynamics of a biological neural network, and that predictable stimuli reinforces existing connections between neurons.

In some embodiments, neurons 135 are trained without using any reward or punishment stimulus. There may be a steady or periodic stream of signals representative of or associated with the virtual environment 155 to neurons 135 during standard operation. Each set of signals may include analog signals delivered to appropriate electrodes 130 (or light emitting elements or chemical emitters) to apply the optical, chemical and/or electrical impulses at specified coordinates and/or with specified intensity/amplitude, frequency/wavelength, and so on. For each set of signals, the neurons 135 may generate responses (e.g., by generating electrical impulses/signals, fluorescing, emitting chemical compounds, etc.). If the signals generated by the neurons 135 corresponds to a target (e.g., is within a target range), then the stream of signals associated with the virtual environment may continue. However, if the signals generated by the neurons 135 does not correspond to the target, then the stream of signals associated with the virtual environment may be paused for a period (e.g., 1-5 seconds), thus depriving the neurons 135 of any stimulus. Accordingly, the system may cease to deliver a stimulus to the in vitro biological neurons for a time period responsive to their output signals failing to satisfy a criteria to elicit self-organizing behavior of the plurality of in vitro biological neurons in a manner that causes the plurality of in vitro biological neurons to interact with or modify the virtual environment or the physical environment. Experimentation has shown that neurons effectively desire a stimulus, and will operate in a manner to increase the chance of receiving a stimulus. Accordingly, neurons can be trained to perform tasks by depriving the neurons of stimuli when they fail to act as desired. This is a different paradigm of learning from reinforcement learning, because in these embodiments there may be no explicitly set reward signals or punishment signals.

Responsive to receiving the training signal (which may be a reward signal or a punishment signal), MEA interface 150 may determine an optical, chemical or electrical stimulation that acts as a reward or punishment or chemical administration that acts as a reward for the biological neurons 135 and/or may send an instruction to the MEA 105 to output the electrical or optical simulation (e.g., reward or punishment stimulus) and/or the chemical administration that acts as a reward. Alternatively, the MEA interface 150 may determine whether to continue providing stimuli associated with the virtual environment or to stop providing stimuli associated with the virtual environment for a time. The integrated circuit 145 may receive the instruction to output the reward or punishment stimulus (or to continue or stop providing stimuli associated with the virtual environment), and may then cause the reward or punishment stimulus to be output to the biological neurons 135 (or may permit stimuli associated with the virtual environment to continue or stop stimuli associated with the virtual environment from being delivered to the neurons 135).

The gap (‘surprise’) between a generated model and observed data may be improved (i.e., minimized) in two ways. Firstly, by the brain either by optimizing probabilistic beliefs about the variables in the generative model or secondly, by acting on the world, such that it is more consistent with the internal generative model. This implies a common objective function (i.e., the variational free energy) for action and perception that scores the fit between an internal model and the world. The gap between the internal generative model and the world is called the surprise which in Bayesian statistic is equivalent to the (log) model evidence. Variational free energy is simply the lower bound on this model evidence. To summarize; if a system of cells, such as neurons 135, holds beliefs about the state of the world, it should continuously update these beliefs to minimize the variational free energy. Thus, a system in which responses result in surprise through unpredictable stimulus should self-organize activity to limit this unpredictable stimulus. Use of unpredictable stimulus to train the neurons 135 is described above. For this work, it is insufficient for a collection of cells to only have their output captured. The cells should also be able to influence the world in some manner with the effects of the actions be observable; in other words, to be embodied in a closed-loop system.

In one embodiment, the reward or punishment stimulus is an electrical stimulus that may be delivered via the array of electrodes 130. For example, a reward stimulus may be an electrical impulse having a delta waveform. The electrical impulse having the delta waveform may be applied at multiple electrodes (e.g., at all of the electrodes in some embodiments) to deliver the electrical impulse to multiple locations in the array (e.g., in the 2D or 3D grid) to provide a deltoid stimulation to the biological neurons 135.

In one embodiment, to prevent forcing hyperpolarized cells from firing, 75 mV at 4 Hz was chosen as the sensory stimulation voltage (e.g., that may relate to where a ball is relative to a paddle in the Pong example). In order to add unpredictable external stimulus into the system, when the culture fails to line the paddle up to connect with the ball, a ‘punishing’ stimulus may be set with an increased variability in the voltage and/or frequency. It is hypothesized that this higher voltage would be sufficient to force action potentials in cells subjected to the stimulation regardless of the state the cell was in, thereby being even more disruptive to the culture.

In one embodiment, a reward stimulus is a chemical reward stimulus. The MEA 105 may further include or be connected to one or more light sources that can emit light of a particular wavelength. These light sources can be activated by the integrated circuit 145 in embodiments. Additionally, the recording chamber 140 may include a protein disposed therein that is sensitive to the particular wavelength of light. The protein (e.g., an opsin protein) may be bound with dopamine or another compound or substance. When the protein is exposed to the particular wavelength of light, the protein may release some amount of the bound dopamine or other compound or substance.

In one embodiment, the reward stimulus includes tetanic stimulation of one or more neurons. A tetanic stimulation includes a high-frequency sequence of individual stimulations of a neuron (or group of neurons). In one embodiment, the high-frequency stimulation comprises a sequence of individual stimulations of one or more neurons delivered at a frequency of about 100 Hz or above. In one embodiment, the high-frequency stimulation comprises a sequence of individual stimulations of one or more neurons delivered at a frequency of about 100 Hz or above. High-frequency stimulation causes an increase in release called post-tetanic potentiation. The presynaptic event is caused by calcium influx. Calcium-protein interactions then produce a change in vesicle exocytosis. The result of such changes causes the postsynaptic neuron to be more likely to fire an action potential.

The chemical reward stimulus, electrical reward stimulus and tetanic stimulation form of reward stimulus all provide a form of reinforcement learning for the biological neurons 135. The punishment stimulus may also provide a form of reinforcement learning. The biological neurons 135 are rewarded when they generate electrical signals that satisfy some criteria of the virtual environment 155 and/or punished when they generate electrical signals that fail to satisfy the criteria, and over time will learn what the targets are and learn how to achieve those targets. The biological neurons 135 may be self-organizing, and may form connections to achieve the targets. In one embodiment, with each success of the biological neurons 135, the chemical or electrical reward stimulus is reduced (e.g., the amount of dopamine released is reduced). In one embodiment, the neurons 135 may learn via Hebbian learning. For example, if two neurons fire together to make something happen and are rewarded, then the next time it takes less activation or voltage to get those two neurons to fire again, thus increasing the frequency of this happening.

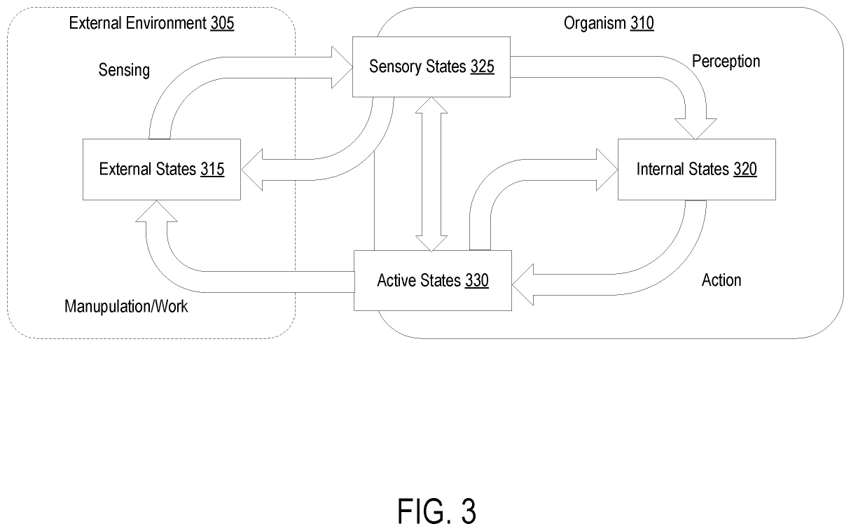

FIG. 3 illustrates a summary of the free energy principal (FEP), whereby an organism 310 will exist in key states, modifying the priors of those states to predict and manipulate an external environment 305 to minimize surprise. Here an external state 315 is sensed by the organism 310 via a sensory state 325 which in turn leads to a given internal state 320. The results of the internal state 320, in conjunction with the sensor state 325, give rise to an active state 330 that can eventually impact the external state 315. It should be noted that external state 315 can be defined as an environment external to the other states and which may or may not be influenced by the active state 330. Moreover, an exchange may occur between active state 330 and sensory state 325 as the organism 310 is aware of its own actions taken, thereby causing sensory state 325 to be a probabilistic outcome influenced by both external state 315 and active state 330. In one example this could constitute a biological neural network, including but not limited to a human brain, receiving information about an external, simulated environment through electrophysiological stimulation. The biological neural network receives information (to internal state 320) both from sensing (e.g., via sensory state 325) the electrophysiological stimulation and receiving information from internally generated (electrophysiological) action potentials (e.g., via active state 330). The action of these neurons can then be applied to the external simulated world via active state 330 and can cause an action which alters the incoming information about the external world at external state 315, which is received by the sensory state 325, thereby continuing the cycle. If the neuron displays a modified internal state 320 to modify the active states 330 to better predict or control the future incoming stimulation from the simulated external world (external environment 305) to decrease information entropy entering the system, this would be consistent with FEP. Likewise, if the biological neural network receives unpredictable or surprising information from external state 315 via sensory state 325 following actions from active state 330 then it would accord with FEP for the organism to modify a future active state 330 or the prior expectations of internal state 320 to decrease the surprise in the future. In another example, a single neuron (e.g., internal state 320 of the single neuron) may receive chemical information (e.g., from active state 330) from an external group of neurons (e.g., external state 315). The neuron (e.g., internal state 320 of the neuron) may react to this incoming information through intracellular or extracellular mechanisms (e.g., via active state 330) which could then impact how the neuron senses (e.g., via sensory state 325) future incoming information (from external state 315) and/or impacts the external state 315 directly.

Biological self-organization has been found at multiple levels, both at the level of the brain and in the neuron. Self-organized neural networks have been observed to form in the neurons 135 on the MEA(s) 105 in embodiments. An innate feature of biological neural networks is the stability of the activity patterns between cells, despite constant external perturbations and ongoing internal processes. This stability, called homeostatic plasticity, has been found to be a canonical feature of neural encoding. It arises from a balance between inhibitory and excitatory activity in the system. Compelling evidence supports that these neural systems display a network state referred to as ‘criticality’, which exist as the set-point where these systems operate. A system in a critical state and non-equilibrium (steady state) with the external environment would maximize both the information capacity and transmission.

Distinct types of criticality in the brain have been observed, with cortical and motor networks operating via different, yet compatible, models of criticality. A theory for how neural networks maintain a state of criticality is through exploiting the free energy principle (FEP). Neurons can perform blind-source separation via a state-dependent Hebbian plasticity that is consistent with the FEP. The FEP proposed that a self-organising system at nonequilibrium steady state with its environment must minimize its variational free energy. In this manner, the brain at spatial and temporal scales engages in active inference by using an internal generative model to predict incoming sensory data. In this way the brain is proposed to act as a Bayesian inference machine.

In response to the application of the electrical, chemical and/or optical impulses at the specified coordinates, one or more of the biological neurons 135 in the biological neural network in the recording chamber 140 will generate an electrical, chemical and/or optical signal. The electrodes 130 may be used as sensors to measure electrical signals that may occur at various coordinates within the array (e.g., the 2D or 3D grid of electrodes 130). For example, the integrated circuit 145 (e.g., a CMOS chip) may read electrical impulses received at the electrodes 130. Alternatively, separate sensors may be arranged in the recording chamber 140. Electrical signals, chemical signals, and/or optical signals output by the neurons 135 may be measured, and their coordinates may be associated with the measurements. Other information such as amplitude (e.g., voltage), intensity, concentration, current and/or frequency may also be measured. The integrated circuit 145 may then generate a digital representation of the one or more measured electrical signals (e.g., using a ADC). This process may be referred to as decoding. This digital representation may then be sent from the MEA 105 to the MEA interface 150.

When one or more biological neurons 135 in the biological neural network generate a signal (e.g., an electrical, optical and/or chemical signal), in some circumstances this may cause one or more nearby biological neurons to also generate a electrical signal. In an example, electrical signals of the one or more nearby biological neurons may or may not trigger still further biological neurons to also generate an electrical signal, which may trigger activity of still more neurons, and so on. Experimental recordings from groups of neurons have shown bursts of activity, so-called neuronal avalanches, with sizes that follow a power law distribution. In neuroscience, the critical brain hypothesis states that certain biological neural networks work near phase transitions. According to this hypothesis, the activity of the brain (or biological neural networks generally) transitions between two phases, one in which activity will rapidly reduce and die, and another where activity will build up and amplify over time. In neuro criticality, the biological neural network capacity for information is enhanced such that subcritical, critical and slightly supercritical branching processes may describe how biological neural networks function. Neuro criticality (which may have a target neuro criticality value) refers to the value or point of the phase transition. The point of the phase transition is the amount of activity that is at a tipping point, below which damping forces prevail (and neural activity quickly dies out), and above which reinforcement forces prevail (and there is an exponential explosion of activity). Neuro criticality implies that on average each time a neuron fires (e.g., generates an electrical signal), this causes on average one other neuron to also fire. However, some inputs (that are above the target neuro criticality value) can cause cascades of activity while other inputs (that are below the target neuro criticality value) can cause very little activity.

In embodiments, one or more neuro criticality values of a biological neural network are measured. Such neuro criticality values may be measured by measuring electrical activity of the neurons over time and performing statistical analysis of the neural activity. These measured neuro criticality values may then be used to enhance, predict, and/or achieve computation on a device. For example, statistical markers for neuro criticality in a biological neural network may be determined by analyzing electrical activity of the biological neural network. For example, electrical activity information may be input into processing logic that performs statistical analysis on the electrical activity information to identify cascades of electrical activity, determine distributions of electrical activity, determine how long the cascades last, determine paths formed by chains of firing neurons, and so on. Such information may be used to determine a neuro criticality value of a biological neural network.

In embodiments, there may be a target neuro criticality value for a biological neural network. If a measured neuro criticality value is below a target criticality value, then the biological neural network may be determined to be below criticality. If the measured criticality value is above the target neuro criticality value, then the biological neural network may be determined to be above criticality. Being either above or below criticality can impair the functioning of the biological neural network. Accordingly, an ability to measure the criticality value of the biological neural network and determine whether it is at, above, or below criticality (e.g., a target neuro criticality value) can be useful in assessing cognitive function of the biological neural network.

In embodiments, one or more other measures of neural activity may also be measured and used to enhance, predict and/or achieve computation on a device. Such other measures may measure information content, complexity, entropy, or a combination thereof. Any such measures may be used separately or together with neuro criticality in embodiments.

Returning to FIG. 1, the digital input signal, instructions for electrical, chemical or optical impulses, representation of electrical, chemical and/or optical signals and/or response messages may be stored in a data store (e.g., for study and/or analysis). Researchers from around the world may access the stored data and/or the virtual environment for study via client computing device 125 connected to the network 120. For example, a researcher may develop an artificial or virtual environment 155 (e.g., a game), run an experiment that applies the game to the neurons 135, and receive data from the experiment. The data from the experiment may then be available to the researcher and/or other researchers via the cloud.

In one embodiment, the server computing device 110 further includes an artificial neural network (e.g., that may be external to virtual environment 155). The artificial neural network may be trained in parallel with the biological neural network comprising the neurons 135. For example, the digital input signal may be input into the artificial neural network, and a target associated with the digital input signal may be provided to the artificial neural network. The artificial neural network may be trained (e.g., using back propagation) at the same time that the biological neural network is trained.

In one embodiment, once neurons 135 are trained to perform a task by virtual environment 155, a score or value may be determined that indicates a level of skill or degree of success of the neurons 135 at performing the task. There are many different types of tasks that the neurons 135 may be trained to perform. What the score or value represents and how it is computed may depend on the type of task or tasks that the neurons 135 were trained to perform. The value may be, for example, a cognitive function value that represents a cognitive function of the biological neural network. In some embodiments, the value is a neuro criticality value and/or is based at least in part on a neuro criticality value. In another embodiment the value is a population based value computed over the entirety of the neural culture or cultures.

One example of a task that the BNN may be trained to perform is the task of playing the computer game Pong. Pong is a simple “tennis-like” game that features two paddles and a ball (though only a single paddle may be modeled in some embodiments). The goal of pong is to defeat an opponent (e.g., which may be a computer opponent provided by the virtual environment, an actual human opponent, or another set of trained neurons) by being the first one to gain 10 points. In Pong, a player receives a point once the opponent misses the ball (which occurs when they fail to move their paddle in front of the ball and allow the ball to move past their paddle to the edge of the screen). The neurons 135 may be trained to perceive the Pong game area, including the moving ball and the two paddles, and to move one of the paddles to intercept the ball. A cognitive function value or other value/score may be determined based on how well the trained neurons play the Pong game. For example, a cognitive function value may be based on a win to loss ratio of the neurons, based on how long the neurons are able to keep the ball in play, based on how many points the neurons can achieve before losing the Pong game, and so on.

In one embodiment, MEA interface 150 determines a cognitive function value for neurons 135. The cognitive function value may be determined based on one or more attempts of the neurons to perform the task that they were trained to perform. In one embodiment, a cognitive function value is determined for each attempt of the neurons 135 to perform the task, and an average cognitive function value is determined based on an average (e.g., a moving average) of the cognitive function values.

In one embodiment, MEA interface 150 determines a neuro criticality value for the neurons 135, as described above. The neuro criticality value may be at or near a target criticality value, and may thus be considered to be at criticality (e.g., at a phase transition). In one embodiment, the cognitive function value is or is based at least in part on the baseline neuro criticality value. In one embodiment, the cognitive function value is distinct from the criticality value. In such embodiments, the cognitive function value and the criticality value may be used together to establish a cognitive function of the neurons 135.

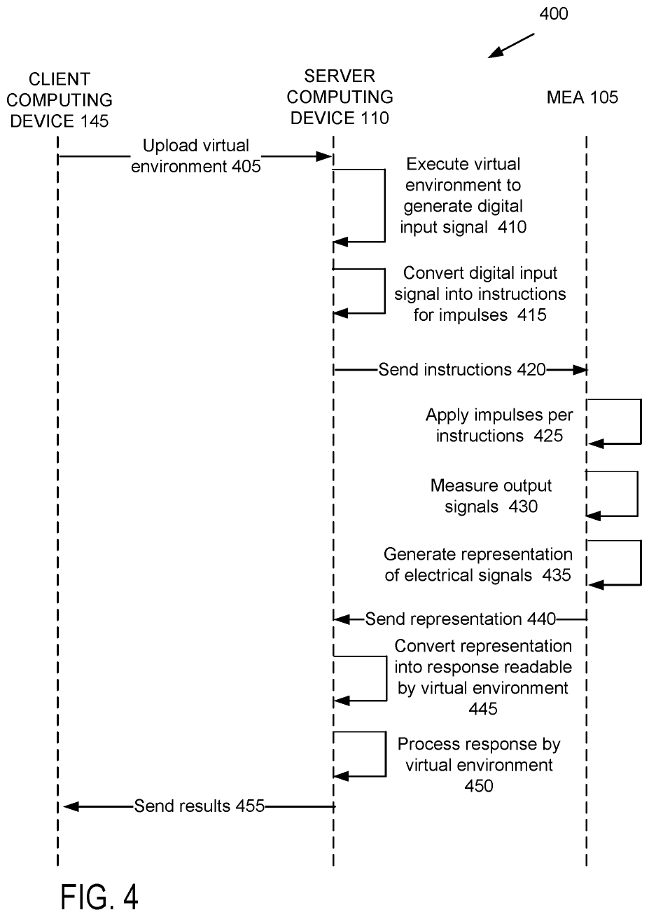

FIGS. 4-5 and 8 are flow diagrams and sequence diagrams illustrating methods of providing a biological computing platform. These methods may be performed by processing logic of a server computing device as well as processing logic of an MEA or similar device (referred to collectively as MEAs for convenience), each of which may include hardware (e.g., circuitry, dedicated logic, programmable logic, microcode, etc.), software (such as instructions run on a processing device, a general purpose computer system, or a dedicated machine), firmware, or a combination thereof. The methods may be performed by an MEA and/or a server computing device. For example, some operations may be performed by an MEA and other operations may be performed by a computing device.

For simplicity of explanation, the methods are depicted and described as a series of acts. However, acts in accordance with this disclosure can occur in various orders and/or concurrently and with other acts not presented and described herein. Furthermore, not all illustrated acts may be performed to implement the methods in accordance with the disclosed subject matter. In addition, those skilled in the art will understand and appreciate that the methods could alternatively be represented as a series of interrelated states via a state diagram or events.

FIG. 4 is a sequence diagram illustrating one embodiment for a method 400 of using a biological computing platform. A client computing device 125 may upload 405 a virtual environment (also referred to as an artificial environment or experiment logic) to a server computing device 110. The virtual environment may be a simulation of a real or physical environment in some embodiments. The server computing device 110 may then execute the virtual environment at block 410, and the virtual environment may generate a digital input signal. An MEA interface executing on the server computing device 110 may receive the digital input signal and convert it into instructions for electrical, chemical and/or optical impulses at block 415. The server computing device 110 may then send the instructions to an MEA 105. In some embodiments, the digital input signal is readable by an integrated circuit (e.g., a processing device) of the MEA 105, and no conversion is performed at block 415. In such embodiments, the digital input signal may be sent to the MEA 105 and processed by the MEA at block 420.

The MEA 105 may generate one or more analog optical, chemical and/or electrical impulses (input signals) based on the instructions (or based on the digital input signal). The signals may be applied at specific electrodes, light sources, chemical emitters, etc. that have specific locations (e.g., x,y coordinates or x,y,z coordinates) at block 425. At block 430, the MEA 105 may then measure output electrical, chemical and/or optical signals generated by biological neurons of a biological neural network in the MEA 105. The MEA may then generate a representation of the electrical, chemical and/or optical signals at block 435. This may include using an analog to digital converter to convert the analog electrical, chemical and/or optical signals into digital values.

At block 440, the MEA sends the representation of the measured electrical, chemical and/or optical signals output by the neurons to the server computing device. At block 445, the MEA interface on the server computing device may convert the representation into a response message for the virtual environment that is readable by processing logic of the virtual environment. The MEA interface may then send the response message to the virtual environment. At block 450, the virtual environment may then process the response message. In some embodiments, the representation of the electrical, chemical and/or optical signals is readable by the virtual environment, and no conversion is performed at block 445. In such embodiments, the representation may be sent to the virtual environment and processed by the virtual environment. The virtual environment may then generate results, which the server computing device 110 may send to the client computing device 125.

In embodiments, the blocks 410-450 form a loop that is continuously run until some stop signal is applied. For example, after the operations of block 450 are completed, the method may return to block 410, and the operations of block 410 may be repeated.

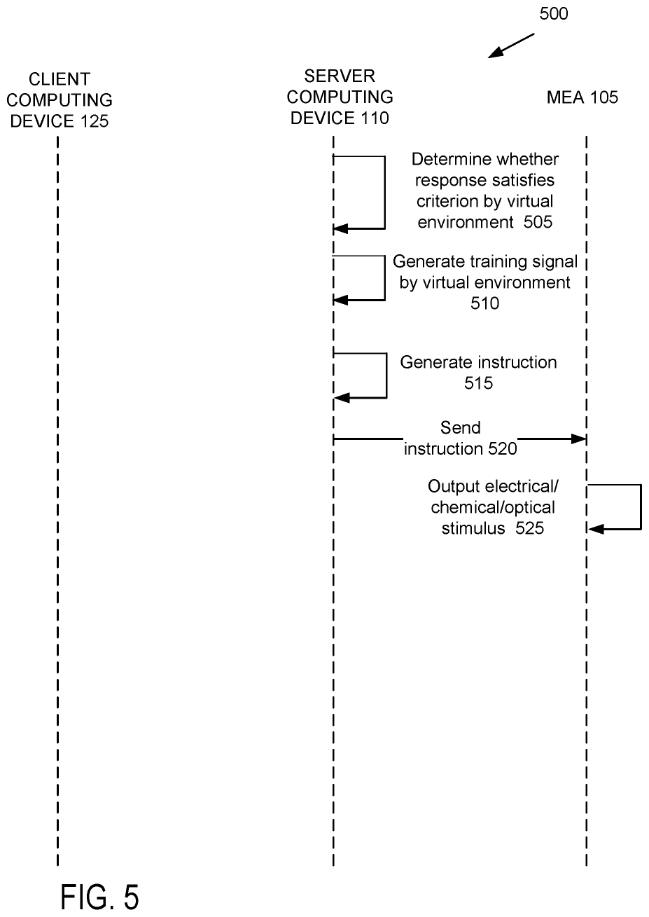

FIG. 5 is a sequence diagram illustrating one embodiment for a method 500 of providing reinforcement learning to biological neurons in a biological computing platform. Method 500 may be performed after method 400 is completed. At block 505, the virtual environment executing on the server computing device 110 determines whether a response message (or representation of electrical, chemical and/or optical signals output by neurons on the MEA 105) satisfies some criterion. If the response message (or representation of electrical, chemical and/or optical signals) satisfies the criterion, then the virtual environment may generate a first training signal and provide the first training signal to the MEA interface executing on the server computing device 110. If the response message (or representation of electrical, chemical and/or optical signals) fails to satisfy the criterion, then the virtual environment may generate a second training signal and provide the second training signal to the MEA interface executing on the server computing device 110. The MEA interface may then generate a reward instruction based on the first training signal or a punishment instruction based on the second training signal (block 515) and send the reward or punishment instruction to MEA 105 (block 520). Alternatively, the MEA interface may forward the training signal to the MEA 105. At block 525, the MEA may then output an electrical, optical and/or chemical reward stimulus or punishment stimulus to the biological neural network on the MEA 105, as appropriate.

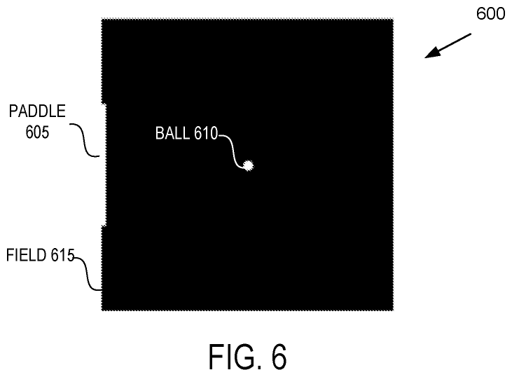

In one example, the virtual environment includes a video game such as Pong. An example of the Pong environment 600 is shown in FIG. 6, which includes a ball 610 and one or more paddle 605 in a field 615. In such an embodiment, the digital input signal may be a projection of the game world (e.g., a frame of a display or user interface of the game). In one embodiment, the projection of the game world is a mapping of the pixels of the display for the game at a given point in time. Each pixel in the display may be associated with a location in a 2D grid of electrodes on the MEA 105. Depending on the resolution of the display and a number of rows and columns in the 2D grid of electrodes, there may be a 1 to 1 mapping between pixels of the display and electrodes in the MEA, a 1 to X mapping or an X to 1 mapping, where X is a positive integer. An MEA interface running on the server computing device 110 may determine a mapping between the pixels of the display for the game and the 2D grid in the MEA 105. For example, x,y pixel 1,3 may map to an electrode at column 2, row 6 of the 2D grid. In one embodiment, converting the digital input signal includes determining, for each location of the 2D grid, whether the location is to be activated (with an impulse sent to the electrode at the location) or deactivated (with no impulse sent to the electrode at the location). Accordingly, the electrical or optical signals may be applied at the specified activated locations.

In the example of Pong, the instructions for electrical/optical impulses may represent a court, a position of a ball and positions of paddles in the court. In this example, the biological neural network may be trained to move the paddle to intercept the ball. Electrophysiological activity of pre-defined motor regions may be recorded to determine how the ‘paddle’ would move, for example. This may be achieved by demarcating the 2D grid in the MEA 105 into 4 quadrants. With each set of electrical/optical impulses that are applied to the biological neural network, the electrical signals generated by the neurons may be measured. If a majority of electrical signals measured are from an upper right quadrant, then this may cause the virtual environment to move the right paddle up. If a majority of electrical signals measured are from a lower right quadrant, then this may cause the virtual environment to move the right paddle down. A positive reward stimulus or other feedback or lack of feedback may then be provided to the biological neural network when the ball intercepts the right paddle, as discussed above.

Given the multitude of possible variations inherent in a system like this, it was beneficial to fix some parameters and empirically test others. In one example, stimulation is delivered at specific locations, frequency, and voltage to key electrodes in a topographically consistent manner in the sensory area relative to the current position of the paddle (e.g., where the virtual environment is the Pong game).

In a broad sense two major ways were proposed to modify performance: encoding of information and decoding of activity, as discussed above. In one embodiment, stimulation in a first motor region may represent an output or command to move a paddle inside of the virtual environment in a first direction, and stimulation in a second motor region may represent an output or command to move the paddle inside the virtual environment in a second direction.

It was hypothesized that the simplified decoding system of measuring activity in two motor regions that were congruent where activity was stimulated (e.g., as set forth in configuration 0 and configuration 1) might not only be inefficient but also prone to bias. To investigate this further an EXP3 machine learning algorithm was used to sample two predefined motor regions to select the best configuration from six possible configurations (e.g., configurations 0-5 shown in FIG. 7) and interpret movement commands for this paddle.

FIG. 7 illustrates an example of multiple different electrode layout schematics of an MEA having a cell culture thereon, including locations of a sensory area that includes stimulation electrodes, multiple motor regions. The motor regions may be divided into one or more first motor regions that perform a first action (e.g., moving a paddle within the virtual Pong environment up) and one or more second motor region that performs a second action (e.g., moving a paddle within the virtual Pong environment down). Representation of example configurations are provided for one example virtual environment, which are described below. In the example electrode layout schematics, stimulation is delivered to a predefined sensory area and activity is measured in multiple motor regions to determine how a paddle will move. Feedback is then provided via the sensory area based on the outcome of the motor area activity. However, it should be understood that the principles discussed with regards to the example of a Pong virtual environment are applicable to a myriad other virtual environments. For example, for environments in which there are more control options or outputs that the neural culture may generate, an electrode layout schematic may include more output regions, which may or may not be motor regions. For example, in a more complicated virtual environment, there may be motor regions for left movement and right movement in addition to up and down movement. Additionally, there may be regions for other types of outputs other than motor or movement outputs.

In embodiments an online optimization method such as EXP3, which may include use of an online machine learning model, is used to select the roles or actions to associate with different outputs of the biological neural network (e.g., neural culture). For example, electrical activity spikes in a first region associated with a first role or first action may be interpreted as an output or instruction to perform the first action. Electrical activity spikes in a second region associated with a second role or second action may be interpreted as an output or instruction to perform the second action. In the example of a neural culture trained to play the game Pong, the online optimization method is used to select one or more first regions for a first motor control and one or more second regions for a second motor control. The online optimization method may start with a discrete set of possible configurations. Each configuration may include a different set of output regions, where each output region is associated with a different output (e.g., action) of the neural culture. The online optimization method performs tests using the different configuration options, and generates scores for each of the options. In one embodiment, the determined scores correspond to the aforementioned cognitive function values. For example, a score may be based on how well a neural culture performs a task that it has been trained to perform. In one embodiment, scores are at least in part based on measured neuro criticality values or other cell specific or population based value or values derived from neural activity. For example, a neuro criticality value may be determined for a particular set of operating conditions and/or a particular configuration. The neuro criticality value may be compared to a target neuro criticality value, which may be associated with a state of criticality. If the neuro criticality value is below the target neuro criticality value (e.g., is below criticality), then the configuration may be assigned a low score.

In addition to testing configurations using the online optimization method, other variables such as type of rewards/punishments used, locations at which stimulation is provided, type of stimulation used, voltages and/or current used, etc. may also be tested using the online optimization method.

The online optimization model may select a configuration to test based on results of tests of that same configuration and/or one or more other configurations in a discrete set of configurations. Alternatively, the online optimization model may randomly generate and test a new configuration in some embodiments. The new configuration may then be added to a list of configurations under consideration. Accordingly, in some embodiments all of the possible configurations to be tested are determined a priori prior running the online optimization model. In other embodiments, no configurations, or a small sample of starting configurations, may be provided to the online optimization model, and the online optimization model may generate multiple different configurations to test. The scores may be continually updated as further tests are run on the various configurations. Ultimately, the configuration having the highest score may be selected for a neural culture.

In some embodiments, a linear decoder is used to select the optimal layout and assignment of different regions or zones (or a continuum of regions or zones) and outputs or roles to each of the regions or zones to the MEA. The linear decoder assigns to each electrode weights associated with one or more different roles or outputs. For example, if there are 5 different outputs, then five different weights may be assigned to an electrode. If there are 2 different outputs, then two different weights may be assigned to an electrode. Assigned weights may be positive values and/or negative values. Online machine learning (e.g., which may include reinforcement learning) may be applied to assign optimal weights to electrodes. Accordingly, the linear decoder may determine optimal roles or outputs to associate with each electrode.

In addition to testing configurations using the linear decoder, other variables such as type of rewards/punishments used, locations at which stimulation is provided, type of stimulation used, voltages and/or current used, etc. may also be tested using the linear decoder.

In one embodiment, an online machine learning model or other online optimization model (e.g., the EXP3 algorithm, other one-arm bandit algorithm, or linear decoder) is used to determine an optimal layout of electrodes for an MEA that enables a neural culture to be trained to perform a task or set of tasks.

Referring to FIG. 7, in embodiments configurations 2-4 were shown to be selected more by the EXP3 algorithm than configurations 0 and 1. Such improved scores for configurations 2-4 is believed to be at least in part due to the phenomenon of lateral inhibition. In embodiments, the layout or configuration of the area of an MEA into input and output regions is selected to take advantage of lateral inhibition of neurons. In biological systems, lateral inhibition is the biological phenomenon in which when one neuron fires, one or more adjacent or lateral neurons of the same type and/or at the same level are inhibited from also firing. Accordingly, in embodiments regions associated with opposing actions or roles (e.g., a first region associated with a motor control to move a paddle up and a second region associated with a motor control to move the paddle down) are arranged adjacent or near to one another. Thus, in embodiments the configuration of the electrodes may be selected to enable lateral inhibition to improve control of biological computing devices. Thus, in the example configurations 2-5, when neurons associated with moving the paddle up fire, this may inhibit the firing of neurons associated with moving the paddle down. Similarly, when neurons associated with moving the paddle down fire, this may inhibit the firing of neurons associated with moving the paddle up.

In embodiments, machine learning and/or lateral inhibition are used to enable an electric system to more efficiently interface with biological activity of a biological system (e.g., a neural culture).

The first configuration (configuration 0) was designed to mimic retinotopic and topographic representations commonly found in nearly all neural systems for representing the external world. Should the system fail to alter activity in the motor regions to move the ‘paddle’ into a correct position to contact the ball, a negative feedback or punishment stimulus (e.g., a random disordered stimulus) may be applied to the neural culture via one or more stimulation electrodes. Alternatively, sensory deprivation may be performed, in which an input signal associated with the virtual environment may be withheld from the neurons for a time period. Other parameters, such as voltage may be determined through empirical testing.

In one example, as shown in configuration 0, two distinct areas were defined as ‘motor regions’, where activity in motor region 1 moved the paddle ‘up’ and activity in motor region 2 moved the paddle ‘down’. In another example, as shown in configuration 1, two distinct areas were defined as ‘motor regions’, where activity in motor region 1 moved the paddle ‘down’ and activity in motor region 2 moved the paddle ‘up’. In another example, as shown in configuration 2, four distinct areas were defined as ‘motor regions’, where activity in motor region 1 moved the paddle ‘up’, and activity in motor region 2 moved the paddle ‘down’, activity in motor region 3 moved the paddle “up” and activity in motor region 4 moved the paddle “down”. In another example, as shown in configuration 4, four distinct areas were defined as ‘motor regions’, where activity in motor region 1 moved the paddle ‘down’, and activity in motor region 2 moved the paddle ‘up’, activity in motor region 3 moved the paddle “down” and activity in motor region 4 moved the paddle “up”. In another example, as shown in configuration 5, four distinct areas were defined as ‘motor regions’, where activity in motor region 1 moved the paddle ‘down’, and activity in motor region 2 moved the paddle ‘up’, activity in motor region 3 moved the paddle “up” and activity in motor region 4 moved the paddle “down”. In another example, as shown in configuration 4, four distinct areas were defined as ‘motor regions’, where activity in motor region 1 moved the paddle ‘up’, and activity in motor region 2 moved the paddle ‘down’, activity in motor region 3 moved the paddle “down” and activity in motor region 4 moved the paddle “up”. In another embodiment not shown, motor regions are not specifically defined and movement is taken based on overall activity across a plurality of neurons without predefined borders based on the relationship of activity between neurons. Different electrode layout configurations were tested to determine an optimal configuration for the given task (e.g., to play the Pong game). The principles set forth herein for determining electrical layout for a neural culture to play the Pong game also apply to how to determine an electrode layout for training a neural culture to perform any other arbitrarily complex task.

In embodiments, the neural culture may not be perfectly symmetrical. For example, there may be more neurons on one side of the MEA than on another side of the MEA. Additionally, regardless of the number of neurons on different regions of the MEA, it may be technically difficult to culture neurons that display perfectly symmetrical electrical activity in different regions (e.g., in the different motor regions shown in FIG. 7). As a result, some motor regions (or other output regions) may exhibit a stronger electrical response than other motor regions (or other output regions) and/or have a higher baseline electrical activity than other motor regions (or other output regions). This may bias the system to detect some types of outputs (e.g., move paddle left) more than other types of outputs (e.g., move paddle right).

Accordingly, in some embodiments a ‘gain’ is added into the system. The system may take a real-time value such as a moving average based on the mean firing (e.g., mean electrical impulse activity) in each motor region (or other output region) over a time period and multiply the mean firing in each motor region (or other output region) by a value to achieve a normalized target value (e.g., a target value of 20 Hz) across the entire region. In embodiments, a target nominal electrical activity may be set, and the moving average electrical activity of each region may be multiplied by a value that achieves the target nominal electrical activity for that region. This would allow changes in activity of each given region to influence the paddle position, even if they displayed different latent spontaneous activity. For example, if a first region has a mean firing of 60 Hz, then electrical signals from that first region may be multiplied by 1/3. If a second region has a mean firing of 10 Hz, then electrical signals from the second region may be multiplied by 2. This effectively normalizes the electrical activity from different regions so that there is no bias for a particular output or action by the neural culture.

Accordingly, in embodiments there exists an uneven distribution of the in vitro biological neurons disposed on a device that at least one of generates first signals that are delivered to neurons or detects second signals based on excitation of neurons. Processing logic may determine, for each region of the device, a respective gain to apply to signals generated by the in vitro biological neurons at the region based on the uneven distribution of the in vitro biological neurons. The processing logic may then, for each region, apply the respective gain associated with the region to those of the second signals that were generated by the region. This effectively normalizes the signals output by the neurons at different regions.

In one embodiment, there are upper and lower thresholds for scaling the electrical signals. For example, electrical signals in a region may not be multiplied by more than 4 or divided by more than 4 in an embodiment. One reason for this is that multiplying by too large a value may decrease a signal to noise ratio of the system below a lower SNR threshold.

In one embodiment, a background electrical activity is determined for each output region (e.g., for each motor region). The background electrical activity may be determined using a moving average. The background electrical activity for a region may then be subtracted from the current electrical activity for that region to determine a normalized electrical activity for the region.

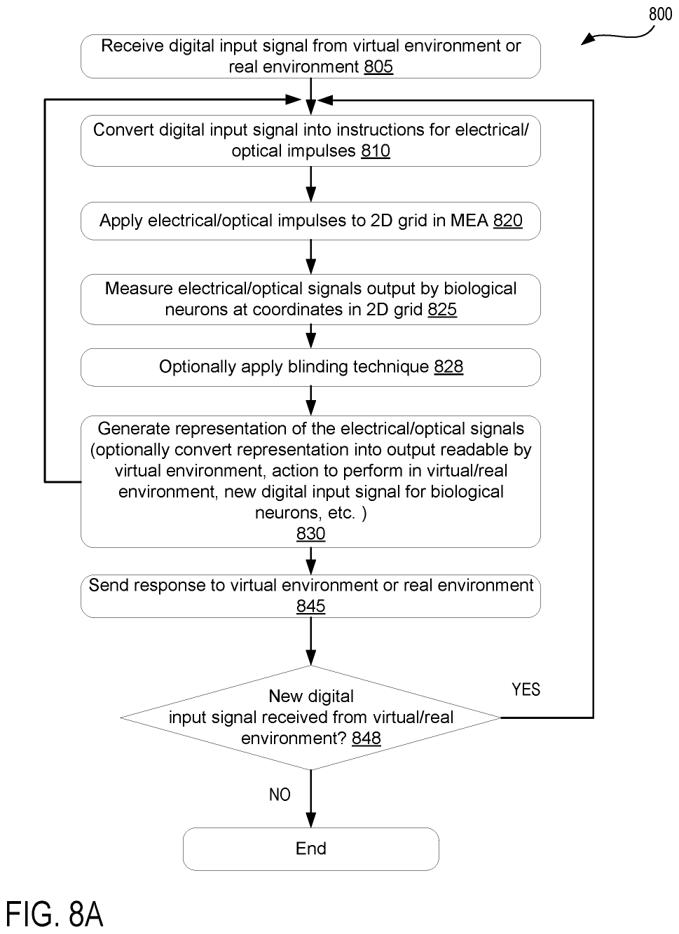

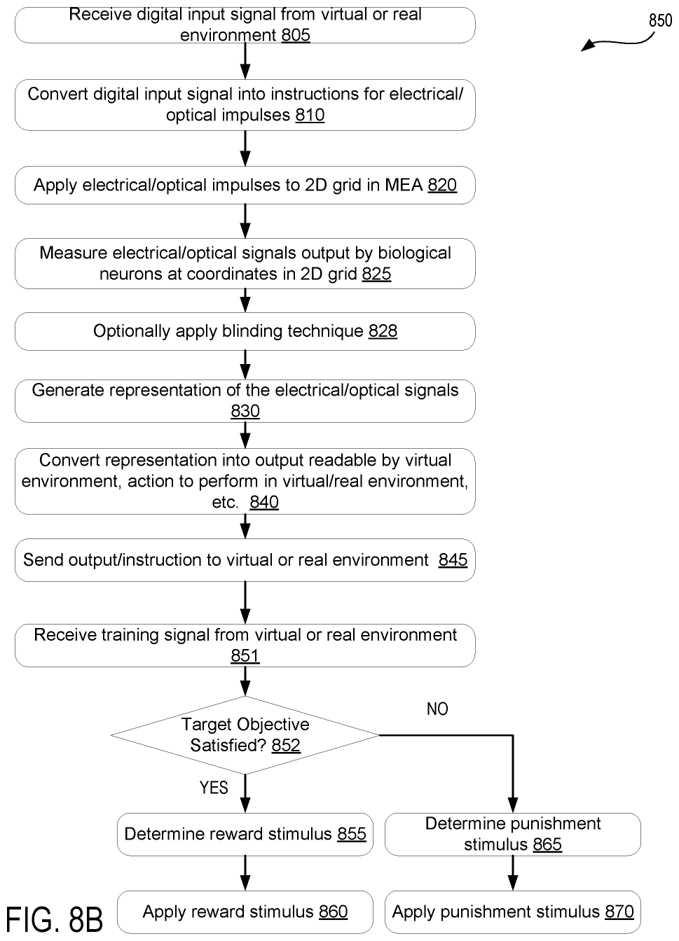

FIG. 8A is a flow diagram illustrating one embodiment for a method 800 of using a biological computing platform. At block 805, a computing device receives a digital input signal from a virtual environment or from a real environment. The digital input signal may be, for example, a tensor. In some embodiments, the virtual environment is a simulation of a real environment, and may reflect a state of the real (e.g., physical) environment. The digital input signal from the real environment may include or be based on one or more measurements, settings, parameters, etc. of a physical environment in some embodiments.

At block 810, the computing device converts the digital input signal into instructions for electrical and/or optical impulses or signals (e.g., encodes a received tensor). The digital input signal is converted into instructions for electrical, chemical and/or optical impulses or signals according to an encoding scheme, which may be a place-based encoding scheme, a time-based encoding scheme, or a hybrid encoding scheme that combines place-based encoding and time-based encoding. In some embodiments, the digital input signal is converted into instructions for chemical impulses instead of or in addition to instructions for electrical and/or optical impulses.

In one embodiment, the encoding is performed using a rate-based coding scheme. In such an embodiment, the encoding comprises determining one or more frequencies at which to apply the electrical, chemical and/or optical signals based on the state of the virtual environment or the real environment. In one embodiment, the encoding is performed using a place-based coding scheme. In such an embodiment, the encoding comprises determining one or more position at which to apply the electrical, chemical and/or optical signals based on the state of the virtual environment or the real environment. In one embodiment, the encoding is performed using a mixed coding scheme that combines a rate-based coding and a place-based coding scheme. In such an embodiment, the encoding comprises determining one or more frequencies at which to apply electrical, chemical and/or optical signals and one or more position at which to apply the electrical, chemical and/or optical signals based on the state of the virtual environment or the real environment.

At block 820, the computing device provides the instructions to an MEA, which applies impulses to an array of electrodes (e.g., a 2D grid or 3D matrix of electrodes), to one or more light sources, to one or more chemical generators/emitters, etc. to cause the electrodes, light sources, chemical emitters, etc. to generate electrical impulses, optical (e.g., light) impulses, and/or chemical impulses. In one embodiment, the encoding is performed at the MEA. In one embodiment, optical signals/impulses are generated using the one or more light sources. These optical signals may cause pores in membranes of the one or more of the plurality of in vitro biological neurons to open, resulting in a change in relative current flow through the membranes. Alternatively, or additionally, the optical signals may stimulate genetically encoded current generators (GECG) in the one or more of the plurality of in vitro biological neurons to generate a voltage. A GECG would be a light sensitive protein or even cell that that undergoes a change when impacted with one or more light sources thereby changing the current in a given direction following the stimulation. In one embodiment, the optical signals stimulate changes in cell membrane characteristics of the one or more of the plurality of in vitro biological neurons via light-based manipulation of at least one of ion channels (e.g., causing the ion channels to open or close), proteins (e.g., causing the proteins to activate, cleave, inhibit, etc.), intra-membrane structures, extra-membrane structures, or trans-membrane structures, and so on. In embodiments, the optical, chemical and/or electrical signals stimulate one or more cells of the plurality of in vitro biological neurons to modify at least one of an electrophysiological property or a somatic property of the one or more cells.

At block 825, the MEA measures electrical signals, chemical signals and/or optical signals output by biological neurons at coordinates of the array (e.g., at coordinates of the 2D grid or 3D matrix). The electrical, chemical and/or optical signals may be analog signals in embodiments.

In one embodiment, at block 828 the MEA or the computing device applies one or more blinding techniques to determine a first subset of the measured electrical, chemical and/or optical signals to process and a second subset of the measured electrical and/or optical signals to ignore. The MEA or computing device may then filter out, ignore, or delete those measured electrical, chemical and/or optical signals in the second subset.

In one embodiment, applying the blinding technique comprises determining an approximate first time at which the input signals were generated and assigning those signals from outputs signals that were generated based on firing neurons within a time window associated with the approximate first time to the second subset. In one embodiment, applying the blinding technique comprises determining magnitudes of each of the outputs signals and assigning those signals from the output signals that have a magnitude that exceeds a threshold to the second subset. In one embodiment, applying the blinding technique comprises determining, for each signal of the outputs signals, a number of electrodes that detected the signal and assigning those signals from the output signals that have were detected by a threshold number of electrodes to the second subset. Other blinding techniques may also be used, such as any of the other blinding techniques described herein.

At block 830, the MEA may generate a digital representation of the electrical signals and/or optical signals. The MEA may send the digital representation to the computing device, which may convert the digital representation into a response message for the virtual environment, into an action to be performed in or on the real environment or virtual environment, to parameters or settings for a device (e.g., a device in a real environment), and so on. Generating the digital representation may include converting the measured electrical, chemical and/or optical signals into the digital representation using an analog to digital converter (e.g., a physical or virtual analog to digital converter). In one embodiment, the electrical, chemical and/or optical signals are converted to a digital representation, which is then processed according to a decoding scheme to determine an output for sending to a computing device. Such decoding may also be performed at the computing device. In one embodiment, the decoding scheme is used to generate the digital representation from the electrical, chemical and/or optical signals. In either instance, the decoding scheme may be a place-based encoding scheme, a time-based encoding scheme, or a hybrid encoding scheme that combines place-based encoding and time-based encoding. The decoding scheme may be a same coding scheme as used for the encoding, or may be an entirely different coding scheme than that used for the encoding. As a simplistic example, a place-based coding scheme may be used for encoding and a time-based coding scheme may be used for decoding.

In one embodiment, the decoding is performed using a rate-based coding scheme. In such an embodiment, the decoding comprises determining one or more frequencies of the measured electrical, chemical and/or optical signals and determining a response to be sent to the virtual environment, an action to be performed in the virtual environment, an action to be performed in the real environment, etc. based at least in part on the one or more frequencies of the measured electrical and/or optical signals.

In one embodiment, the decoding is performed using a place-based coding scheme. In such an embodiment, the decoding comprises determining one or more positions of the measured electrical, chemical and/or optical signals and determining a response to be sent to the virtual environment, an action to be performed in the virtual environment, an action to be performed in the real environment, etc. based at least in part on the one or more positions of the measured electrical, chemical and/or optical signals.

In one embodiment, the decoding is performed using a mixed coding scheme that combines a rate-based coding and a place-based coding scheme. In such an embodiment, the decoding comprises determining one or more frequencies of the measured electrical, chemical and/or optical signals, determining one or more positions of the second signals, and determining a response to be sent to the virtual environment, an action to be performed in the virtual environment, an action to be performed in the real environment, etc. based at least in part on the one or more positions of the measured electrical, chemical and/or optical signals and the one or more frequencies of the measured electrical, chemical and/or optical signals.

In embodiments, the first subset of the measured electrical, chemical and/or optical signals are decoded without decoding the second subset of the measured electrical, chemical and/or optical signals.

The method may then proceed to block 845, at which the computing device may provide the response message, action, settings, parameters, etc. to the virtual environment and/or real (e.g., physical) environment. In some embodiments, at block 830 the representation is additionally or alternatively converted into an updated digital input signal that will act as a future stimulus (e.g., new tensor) to be provided back to the neurons. Accordingly, in embodiments the method returns to block 810 and the updated digital signal generated to block 830 is converted into instructions for new electrical and/or optical and/or chemical impulses. In some embodiments, at block 830 the representation of the electrical, chemical and/or optical signals is compared to a target, and an error is determined based on a different between the target and the representation of the electrical, chemical and/or optical signals. The error may then be used to generate the updated digital signal, which can then be converted into new instructions or electrical, optical and/or chemical impulses at block 810. Accordingly, in embodiments a closed-loop feedback system is provided.

At block 848, the real or virtual environment may update its state based on the output generated at block 830. The real or virtual environment may then generate a new input signal based on its updated state. This may include determining a future stimulus for the neurons, and generating a new tensor representative of the future stimulus. If a new digital input signal is not generated by the environment, then the method may end. If a new digital input signal is generated by the environment, then the method may return to block 810, and the new digital input signal may be converted into instructions for electrical, optical and/or chemical impulses.

FIG. 8B is a flow diagram illustrating one embodiment for a method 850 of using a biological computing platform. Method 850 may include some or all of the operations discussed with reference to method 800, and may include one or more additional operations directed to training.

In one embodiment, at block 805, a computing device receives a digital input signal from a virtual environment or from a real environment. In some embodiments, the virtual environment is a simulation of a real environment, and may reflect a state of the real (e.g., physical) environment. The digital input signal from the real environment may include or be based on one or more measurements, settings, parameters, etc. of a physical environment in some embodiments.

At block 810, the computing device converts the digital input signal into instructions for electrical, chemical and/or optical impulses or signals. The digital input signal is converted into instructions for electrical, chemical and/or optical impulses or signals according to an encoding scheme, which may be a place-based encoding scheme, a time-based encoding scheme, or a hybrid encoding scheme that combines place-based encoding and time-based encoding. In some embodiments, the digital input signal is converted into instructions for chemical impulses instead of or in addition to instructions for electrical, chemical and/or optical impulses.

In one embodiment, the encoding is performed using a rate-based coding scheme. In such an embodiment, the encoding comprises determining one or more frequencies at which to apply the electrical, chemical and/or optical signals based on the state of the virtual environment or the real environment. In one embodiment, the encoding is performed using a place-based coding scheme. In such an embodiment, the encoding comprises determining one or more position at which to apply the electrical, chemical and/or optical signals based on the state of the virtual environment or the real environment. In one embodiment, the encoding is performed using a mixed coding scheme that combines a rate-based coding and a place-based coding scheme. In such an embodiment, the encoding comprises determining one or more frequencies at which to apply electrical, chemical and/or optical signals and one or more position at which to apply the electrical, chemical and/or optical signals based on the state of the virtual environment or the real environment.

At block 820, the computing device provides the instructions to an MEA, which applies the electrical, chemical and/or optical impulses to an array of electrodes (e.g., a 2D grid or 3D matrix of electrodes), to one or more light sources, to one or more chemical generators/emitters, etc.

At block 825, the MEA measures electrical signals, chemical signals and/or optical signals output by biological neurons at coordinates of the array (e.g., at coordinates of the 2D grid or 3D matrix). The electrical, chemical and/or optical signals may be analog signals in embodiments.

In one embodiment, at block 828 the MEA or the computing device applies one or more blinding techniques to determine a first subset of the measured electrical, chemical and/or optical signals to process and a second subset of the measured electrical and/or optical signals to ignore. The MEA or computing device may then filter out, ignore, or delete those measured electrical, chemical and/or optical signals in the second subset.

At block 830, the MEA may generate a digital representation of the electrical signals, chemical signals and/or optical signals. This may include using a physical or virtual analog to digital converter to convert analog electrical, chemical and/or optical signals output by neurons and detected by electrical, chemical and/or optical sensors into a digital signal.

At block 840, the MEA may send the digital representation to the computing device, which may convert the digital representation into an output understandable to the real or virtual environment. This may generating an output or instruction for the virtual or real environment based on the digital representation according to a decoding scheme, which may be a place-based encoding scheme, a time-based encoding scheme, or a hybrid encoding scheme that combines place-based encoding and time-based encoding. The decoding scheme may be a same coding scheme as used for the encoding, or may be an entirely different coding scheme than that used for the encoding. As a simplistic example, a place-based coding scheme may be used for encoding and a time-based coding scheme may be used for decoding.

At block 845, the computing device may provide the output or instruction (e.g., response message, action, settings, parameters, etc.) to the virtual environment and/or real (e.g., physical) environment.

At block 851, the computing device may receive a training signal from the virtual environment or real environment. The training signal may indicate whether the output or instruction caused the virtual environment or real environment to reach a target state or condition or to otherwise satisfy some condition or criteria. At block 852, processing logic may determine whether one or more criteria (e.g., a target objective) was satisfied and/or whether the training signal is a reward (positive reinforcement) signal or a punishment (negative reinforcement) signal. If the objective was satisfied and/or the training signal was a reward signal, the method continues to block 855. If the objective was not satisfied and/or the training signal was a punishment signal, the method continues to block 865.

At block 855, the computing device may determine an electrical, optical or chemical reward stimulus (e.g., a predictable stimulus) and instruct the MEA to apply the reward stimulus. The reward stimulus may include a continuation of stimuli to the neurons, a particular electrical, optical and/or chemical stimulus, and so on, as discussed elsewhere herein. Alternatively, the computing device may forward the training signal to the MEA, which may then determine a reward stimulus to provide. At block 860, the MEA then applies the reward stimulus to the biological neurons.

At block 865, the computing device may determine an electrical, optical or chemical punishment stimulus (e.g., an unpredictable stimulus) and instruct the MEA to apply the punishment stimulus. The punishment stimulus may include a cessation of stimuli to the neurons, a particular electrical, optical and/or chemical stimulus, and so on, as discussed elsewhere herein. In one embodiment, the MEA ceases to deliver a stimulus to the in vitro biological neurons for a time period responsive to the target object not being satisfied to elicit self-organizing behavior of the plurality of in vitro biological neurons in a manner that causes the plurality of in vitro biological neurons to interact with or modify the virtual environment or the physical environment. In one embodiment, the computing device may forward the training signal to the MEA, which may then determine a punishment stimulus to provide. At block 870, the MEA then applies the punishment stimulus to the biological neurons.

This process may continue, and in turn a biological neural network may be trained. Training may be ongoing, and may be performed while the biological neural network is being used in the field in embodiments. In some embodiments, the operations of method 800 and the operations of method 850 are combined.

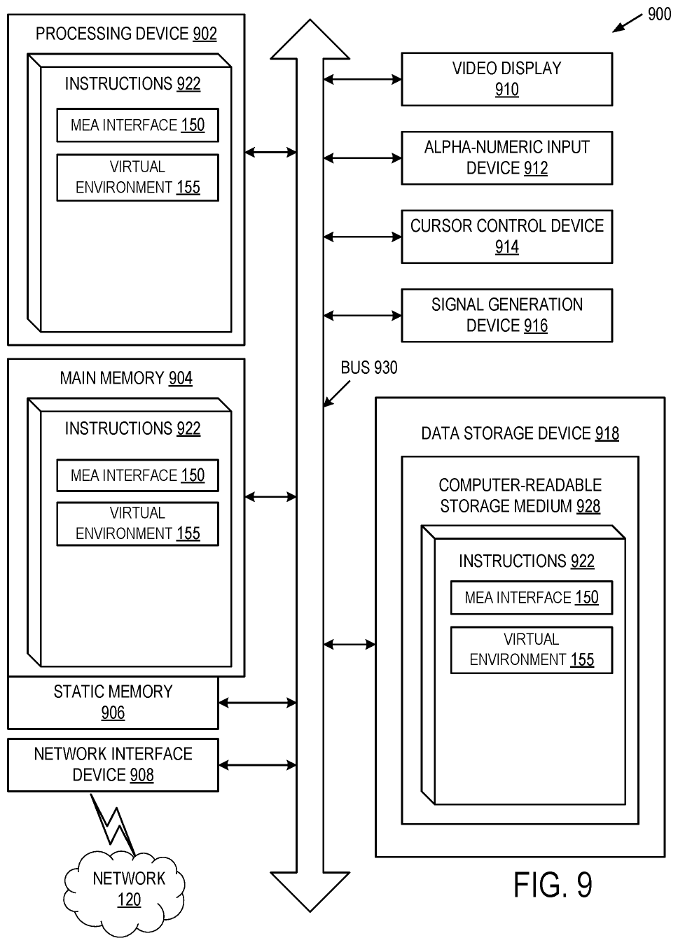

FIG. 9 illustrates a diagrammatic representation of a machine in the example form of a computing device 900 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein.

The example computing device 900 includes a processing device 902, a main memory 904 (e.g., read-only memory (ROM), flash memory, dynamic random access memory (DRAM) such as synchronous DRAM (SDRAM) or Rambus DRAM (RDRAM), etc.), a static memory 906 (e.g., flash memory, static random access memory (SRAM), etc.), and a secondary memory (e.g., a data storage device 918), which communicate with each other via a bus 930.

Processing device 902 represents one or more general-purpose processors such as a microprocessor, central processing unit, or the like. More particularly, the processing device 902 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 902 may also be one or more special-purpose processing devices such as an application specific integrated circuit (ASIC), a field programmable gate array (FPGA), a digital signal processor (DSP), network processor, or the like. Processing device 902 is configured to execute the processing logic (instructions 922) for performing the operations and steps discussed herein.

The computing device 900 may further include a network interface device 908. The computing device 900 also may include a video display 910 (e.g., a liquid crystal display (LCD) or a cathode ray tube (CRT)), an alphanumeric input device 912 (e.g., a keyboard), a cursor control device 914 (e.g., a mouse), and/or a signal generation device 916 (e.g., a speaker).

The data storage device 918 may include a machine-readable storage medium (or more specifically a computer-readable storage medium) 928 on which is stored one or more sets of instructions 922 embodying any one or more of the methodologies or functions described herein. The instructions 922 may also reside, completely or at least partially, within the main memory 904 and/or within the processing device 902 during execution thereof by the computer system 900, the main memory 904 and the processing device 902 also constituting computer-readable storage media.

The computer-readable storage medium 928 may also be used to store MEA interface 150 and/or virtual environment 155 (as described with reference to the preceding figures), and/or a software library containing methods that call MEA interface 150 and/or virtual environment 155. While the computer-readable storage medium 928 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any non-transitory medium that is capable of storing or encoding a set of instructions for execution by the machine and that cause the machine to perform any one or more of the methodologies described herein. The term “non-transitory computer-readable storage medium” shall accordingly be taken to include, but not be limited to, solid-state memories, and optical and magnetic media.

Some portions of the detailed description have been presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, as apparent from the following discussion, it is appreciated that throughout the description, discussions utilizing terms such as “receiving”, “converting”, “sending”, or the like, may refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Embodiments of the present disclosure also relate to an apparatus for performing the operations herein. This apparatus may be specially constructed for the discussed purposes, and/or it may comprise a general purpose computer system selectively programmed by a computer program stored in the computer system. Such a computer program may be stored in a computer readable storage medium, such as, but not limited to, any type of disk including floppy disks, optical disks, CD-ROMs, and magnetic-optical disks, read-only memories (ROMs), random access memories (RAMs), erasable programmable read only memories (EPROMs), electrically erasable programmable read only memories (EEPROMs), magnetic disk storage media, optical storage media, flash memory devices, other type of machine-accessible storage media, or any type of media suitable for storing electronic instructions, each coupled to a computer system bus.

It is to be understood that the above description is intended to be illustrative, and not restrictive. Many other embodiments will be apparent to those of skill in the art upon reading and understanding the above description. Although the present disclosure has been described with reference to specific example embodiments, it will be recognized that the disclosure is not limited to the embodiments described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

What is claimed is:

1. A method of interfacing with a plurality of in vitro biological neurons, comprising:
generating, by a processing device, a first tensor indicative of a state of a virtual environment or a real environment;
encoding the first tensor into a plurality of instructions;
generating first signals according to the plurality of instructions using a first plurality of electrodes, one or more chemical emitters, or one or more light sources to stimulate action potentials in one or more of the plurality of in vitro biological neurons;
detecting second signals by a second plurality of electrodes, one or more chemical sensors, or one or more image sensors, the second signals having been generated by the one or more of the plurality of in vitro biological neurons, wherein the second signals represent an action associated with the virtual environment or the real environment;
decoding the second signals into a second tensor; and
applying the action to the virtual environment or the real environment based on the second tensor.

2. The method of claim 1, wherein the first signals are optical signals generated using the one or more light sources, and wherein the optical signals cause pores in membranes of the one or more of the plurality of in vitro biological neurons to open, resulting in a change in relative current flow through the membranes.

3. The method of claim 1, wherein the first signals are optical signals generated using the one or more light sources, and wherein the optical signals stimulate genetically encoded voltage generators in the one or more of the plurality of in vitro biological neurons to generate a voltage.

4. The method of claim 1, further comprising:
stimulating changes in cell membrane characteristics of the one or more of the plurality of in vitro biological neurons via light-based manipulation of at least one of ion channels, proteins, intra-membrane structures, extra-membrane structures, or trans-membrane structures.

5. The method of claim 1, wherein:
the first signals are optical signals generated using the one or more light sources, the first signals having a wavelength; and
the second signals are optical signals detected using the one or more image sensors, the second signals having been generated by fluorescent reporters, in the one or more of the plurality of in vitro biological neurons, that emit light from excitation of proteins sensitive to the wavelength.

6. The method of claim 1, wherein the first signals are optical signals and the second signals are optical signals.

7. The method of claim 1, wherein the first signals are electrical signals and the second signals are optical signals.

8. The method of claim 1, wherein the first signals are optical signals and the second signals are electrical signals.

9. The method of claim 1, wherein the first signals stimulate one or more cells of the plurality of in vitro biological neurons to modify at least one of an electrophysiological property or a somatic property of the one or more cells.

10. The method of claim 1, wherein the plurality of in vitro biological neurons are disposed on a device, wherein the device is an electrical device, an optical device, or a hybrid device comprising optical and electrical components, and wherein the first plurality of electrodes, the one or more chemical emitters, or the one or more light sources and the second plurality of electrodes, the one or more chemical sensors, or the one or more image sensors are disposed in an array on the device.

11. The method of claim 10, wherein the device comprises a multielectrode array (MEA).

12. The method of claim 1, wherein the plurality of in vitro biological neurons comprise a genetically encoded voltage indicator (GEVI) and a bioluminescence resonance energy transfer (BRET) that fluoresce to generate the second signals.

13. The method of claim 1, wherein the plurality of instructions comprise a plurality of electrical potentials.

14. A system comprising:
a computing device; and
a cell excitation and measurement device operatively connected to the computing device, the cell excitation and measurement device comprising a plurality of in vitro biological neurons disposed thereon, and the cell excitation and measurement device further comprising:
at least one of a plurality of electrodes, a plurality of chemical emitters, or one or more light sources configured to excite the in vitro biological neurons; and
at least one of the plurality of electrodes, a plurality of chemical sensors, or one or more image sensors to measure responses of the plurality of in vitro biological neurons to excitation;
wherein the computing device is to generate a first tensor indicative of a state of a virtual environment or a real environment and encode the first tensor into a plurality of instructions;
wherein the cell excitation and measurement device is to:
generate first signals according to the plurality of instructions using a first subset of the plurality of electrodes, a first subset of the plurality of chemical emitters, or a first subset of the one or more light sources; and
detect second signals using at least one of a second subset of the plurality of electrodes, a second subset of the plurality of chemical sensors, or a second subset of the one or more image sensors, the second signals having been generated by one or more of the plurality of in vitro biological neurons, wherein the second signals represent an action associated with the virtual environment or the real environment; and
wherein the computing device is further to:
decode the second signals into a second tensor; and
apply the action to the virtual environment or the real environment based on the second tensor.

15. The system of claim 14, wherein the cell excitation and measurement device comprises the one or more light sources and the plurality of electrodes, and wherein the first signals are optical signals generated using the first subset of the one or more light sources, and wherein the optical signals cause pores in membranes of the one or more of the plurality of in vitro biological neurons to open, resulting in a change in relative current flow through the membranes.

16. The system of claim 14, wherein the cell excitation and measurement device comprises the one or more light sources and the plurality of electrodes, and wherein the first signals are optical signals generated using the first subset of the one or more light sources, and wherein the optical signals stimulate genetically encoded voltage generators in the one or more of the plurality of in vitro biological neurons to generate a voltage.

17. The system of claim 14, wherein the plurality of in vitro biological neurons are configured such that light-based manipulation of at least one of ion channels, proteins, intra-membrane structures, extra-membrane structures, or trans-membrane structures of the plurality of in vitro biological neurons stimulates changes in cell membrane characteristics of the plurality of in vitro biological neurons.

18. The system of claim 14, wherein the cell excitation and measurement device comprises the one or more light sources and the one or more image sensors, and wherein the plurality of in vitro biological neurons comprise fluorescent reporters that emit light from excitation of proteins sensitive to a wavelength produced by the one or more light sources.

19. The system of claim 14, wherein the cell excitation and measurement device comprises the one or more light sources to generate the first signals and the one or more image sensors to record the second signals.

20. The system of claim 14, wherein the cell excitation and measurement device comprises the plurality of electrodes to generate the first signals and the one or more image sensors to detect the second signals.

21. The system of claim 14, wherein the cell excitation and measurement device comprises the one or more light sources to generate the first signals and the plurality of electrodes to detect the second signals.

22. The system of claim 14, wherein the cell excitation and measurement device comprises a multielectrode array (MEA).

23. The system of claim 14, wherein the plurality of in vitro biological neurons comprise a genetically encoded voltage indicator (GEVI) and a bioluminescence resonance energy transfer (BRET) that fluoresce to generate the second signals.
